(19) United States
(12) Patent Application Publication
(10) Pub. No.: US 2025/0090367 A1
(43) Pub. Date: Mar. 20, 2025

(54) METHODS AND SYSTEMS TO TRACK AND GUIDE IMPROVEMENT IN HEALTH AND FITNESS BIOMETRICS

(71) Applicant: Sleep Solutions of Texas, LLC, Tyler, TX (US)

(72) Inventor: Raghavendra Vitthalrao GHUGE, Tyler, TX (US)

(73) Assignee: Sleep Solutions of Texas, LLC, Tyler, TX (US)

(21) Appl. No.: 18/890,703

(22) Filed: Sep. 19, 2024

Related U.S. Application Data
(60) Provisional application No. 63/583,756, filed on Sep. 19, 2023.

(51) Int. Cl. A61F 5/56; G16H 10/60; G16H 15/00; G16H 20/13; G16H 20/30

(52) U.S. Cl. CPC A61F 5/566; G16H 10/60; G16H 15/00; G16H 20/13; G16H 20/30

(57) ABSTRACT

A system for use with a mandibular lingual repositioning device has a computing application implementable on a user's personal computing device in operative communication with a managing platform over a communication network. The computing application has a first module configured for entry of a user's biometrics, a second module configured for entry of a user's health/fitness goals, a third module configured for calculating a plurality of variables relative to the user's health/fitness goals. The plurality of variables includes carbohydrates daily intake, protein daily intake, fat daily intake, calories per day, calories per week, basal metabolic rate, ml/hr hydration loss during exercise, active metabolic rate, and a target calorie burn rate. The managing platform has a user interface configured for managing the data from the user's computing application, managing access to the user's computing application, and managing one or more algorithms executable by the third module of the computing application.

### TECHNICAL FIELD

This application relates to methods to track and guide improvements in health and fitness biometrics for users wearing or not wearing a mandibular repositioning devices configured to increase that user's smallest concentric airway cross-sectional area size during physical activity.

### BACKGROUND

Many individuals suffer from disordered breathing while asleep and many more have significant narrowing at the level of the smallest concentric airway cross-sectional area (SMCA) size while awake when studied by cone CT scans. Moreover, many individuals may have restricted airway passages but do not know they have them because of lack of symptoms. Also, many individuals may have normal airway size but could benefit from an increase in airway size during athletic or physical activities that demand extreme amounts of oxygen, glucose, adrenaline and other natural chemicals the human body makes or needs. Some example disorders associated with reduced SMCA include obstructive sleep apnea (OSA), snoring, snore arousals, sleep-related hypoxia, and other conditions dependent on and caused by snoring or OSA.

The maximum heart rate that human being is able to attain during physical activity is often given by a formula that is equal to 220 minus the age of the individual (in years). A desired heart rate during exercise for burning fat is found to be in the range of 60% to 70% of the maximum heart rate and while the desired hear rate for cardio activity is 70% to 80% of the maximum heart rate. People with a narrowing of their SMCA will often find themselves breathing much harder during exercise and thus may generate a higher heart rate that is greater than the 60% to 70% range; thus, moving more quickly from the fat burning rate to a cardio rate (70% to 80% range), which may not be desired. These observations are important because the windows for these two exercise goals (fat burning and cardio) are narrow and adjacent to each other, and an individual can very easily be in cardio range while desiring to be in fat burning range.

The SMCA on average is about 149 mm². This is the narrowest point in an adult human airway. Many humans have much smaller airways as shown by cone CT scans of the airway. It has been observed that OSA patients have an SMCA on average of about 40 mm²-67 mm². In OSA, the mandible lowers to a greater degree than in normal sleep due to activation of the upper airway muscles (due to lack of oxygen) allowing traction on hyoid bone and mouth opening to facilitate mouth breathing. However, this lowering of the mandible comes at a price of reducing the antero-posterior diameter of the airway due to posterior movement of the mandible and tongue in the second half of the lowering process (the second 13°). Anterior (sagittal) repositioning of the mandible alone does not counteract this part of physiology. Studies have shown that vertical (caudal) repositioning of the mandible has a greater influence on increasing the transverse diameter of the SMCA than anterior repositioning. Moreover, applicant believes that simultaneously advancing the mandible sagittally while advancing it caudally can mitigate airway narrowing that occurs during voluntary mouth opening in OSA. Both such simultaneous or sequential repositioning increases the AP diameter and transverse diameter of the SMCA simultaneously. These simultaneous increase in AP and transverse diameters effectively incrementally increase the SMCA.

Obesity has become an epidemic across the globe. Pediatric obesity, accounting for 50% of American children, probably will result in a significantly less healthy generation of Americans in years to come. One goal is to equip parents and children (youth and adults alike) to create a healthier world. We want to inspire a generation of health & fitness lovers, improve athletic performance, and reduce poor cardiovascular, neurological, and metabolic outcomes.

There is also a need to track and guide improvements in the athletic performance, sleep, physical fitness, body composition, weight management, hydration, metabolic biometric parameters such as oxygen, blood levels of glucose and other, nutrition, and cardio-pulmonary functions of an individual and to connect the individual with the appropriate professional to monitor and advise the individual regarding the same. The professional may be a trainer, doctor, dentist, a health professional, a sports professional, a fitness professional, a sports franchise, college sports program, high school sports program, weight loss programs and their leaders, or the like. The tracking and guidance can be for an individual in their all-natural state or in an enhanced state whereby the individual is fitted with a mandibular repositioning device to increase the size of their smallest concentric airway cross-sectional area. This increase in the concentric airway cross-sectional area can keep the heart rate lower, which makes breathing easier during physical activity, in particular by moving the mandible forward and downward and moving the tongue forward and one that effectively restores the disrupted natural channels of salivary flow.

### SUMMARY

The mandibular repositioning device introduced herein opens a user's airway, especially increasing the size of a user's smallest concentric airway cross-sectional area, for improvement in numerous aspects of performance during physical activity, especially a lower heart rate, and has effective salivary flow through one or more salivary flow channels in one or both of the mandibular piece and maxillary piece. The methods and devices disclosed herein are able to increase the smallest concentric airway cross-sectional area of any human airway, be it small, average, or large at its original size. The methods and devices are able to advance the mandible and tongue of the user anteriorly and caudally to increase the rate of airflow, decrease the work of breathing, and thereby enhance physical performance of the user, for example, speed, endurance, strength, and accuracy. Individuals with a reduced SMCA will likely see a greater benefit than those with a “normal” SMCA, but both will see benefits.

In all aspects, systems for use with a mandibular lingual repositioning device are disclosed that have a computing application implementable on a user's personal computing device in operative communication with a managing platform over a communication network. The computing application has a first module configured for entry of a user's biometrics, a second module configured for entry of a user's health/fitness goals, a third module configured for calculating a plurality of variables relative to the user's health/fitness goals. The plurality of variables includes carbohydrates daily intake, protein daily intake, fat daily intake, calories per day, calories per week, basal metabolic rate, ml/hr hydration loss during exercise, active metabolic rate, and a target calorie burn rate. The managing platform has a user interface configured for managing the data from the user's computing application, managing access to the user's computing application, and managing one or more algorithms executable by the third module of the computing application.

In another aspect, the mandibular piece has a plateau of a preselected height between the base of the protrusive flange and the tooth covering. The preselected height of the plateau prevents disconnect between each protrusive flange and its respective driver flange relative to a fully open mouth measurement between incisors of the user. In all embodiments, the plateau can extend across the full width of the tooth covering. In one embodiment, the plateau is wedge-shaped, has a first height at the anterior base of the protrusive flange, a second height at the posterior base of the protrusive flange, and the first height is greater than the second height; and wherein the protrusive flange and driver flange are inclined equivalently to the plateau to maintain the engaged convex portion to convex curvature thereof. Here, the plateau extends posteriorly to a posterior terminus of the tooth covering and terminates with a third height that is smaller than the first height and the second height.

In another aspect, mandibular repositioning devices are disclosed that have a maxillary piece comprising a tooth covering having a driver flange protruding laterally outward on a right side proximate a backmost teeth mold and/or on a left side proximate a backmost teeth mold and a mandibular piece comprising a tooth covering having a protrusive flange extending cranially therefrom positioned to have a posterior side engaged with the anterior side of each driver flange. Each driver flange has an anterior side with a convex curvature, and each protrusive flange has a posterior side with a concave-to-convex curvature from its base toward its most cranial point and a convex portion of the concave-to convex curvature engages the convex curvature of the driver flange in a rest position. The downward movement of the mandibular piece moves the convex portion of the posterior side of the protrusive flange along the convex curvature of the driver flange moves the user's mandible forward.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present system.

FIG. 1 is a schematic illustration of a system in operative communication with the MRLD.

FIG. 2 is a side view of a first embodiment of a mandibular repositioning device that provides Dynamic Continuous Open Airway Technology (DCOAT) to the user.

FIG. 3 is a side view of a second embodiment of a mandibular repositioning device that provides Dynamic Continuous Open Airway Technology (DCOAT) to the user.

FIG. 4 is a mathematical model of how to position and determine the convex and concave curvatures of the protrusive flange and the driver flange of a mandibular reposition device.

FIG. 5 is a photograph of a selected patient's mandibular repositioning device having the protrusive flange with the convex to concave curvature described herein against the convex curvature of the driver flange.

FIG. 6 is a left side computer generated image of an embodiment of the mandibular repositioning device in an open position in the mouth of a skull.

FIG. 7 is a comparison of a mandibular repositioning device (left) absent a plateau to a one with a plateau added to the base of the protrusive flange (right).

FIG. 8 is a rear view of the mandibular piece with the mandibular plateau of FIG. 7.

FIG. 9 is a side view of an alternate embodiment for a protrusive flange having a mandibular plateau that is wedge shaped.

FIG. 10 is a side view of the embodiment of FIG. 9 with the mandibular plateau extended to the posterior surface to fill the gap shown in FIG. 9 created between mandibular piece and the maxillary piece.

FIG. 11 is a side view of a maxillary piece having a driver flange that is extended caudally, represented by the dashed lines to fill the gap shown in 9 created between mandibular piece and the maxillary piece.

FIG. 12 is a flowchart of the physiological effects of increasing the size of the SMCA of a user with the mandibular repositioning devices herein.

FIG. 13 is a Table version of Algorithm I from an Excel worksheet showing the equations in the cells.

FIG. 14 is a Table version of Algorithm II from an Excel worksheet showing the equations in the cells and then filled in with one example of data inputs and the corresponding calculated outputs.

FIG. 15 is a flowchart for data entry flow within a health and biometric app for implementation on a personal computing device.

FIG. 16 is first portion of a more detailed flowchart for data entry and data outputs for a portion of the health and biometric app of FIG. 15.

FIG. 17 is a second portion of a more detailed flowchart for data entry and data outputs for a portion of the health and biometric app of FIG. 15.

FIG. 18 is a screen shot flow for the “Set Your Goals” Nutrition portion of the flowchart.

FIG. 19 is a screen shot flow for the “Set Your Goal” Body Composition portion of the flowchart.

FIG. 20 is a screen shot flow for the “Set Your Goal” Hydration portion of the flowchart.

FIG. 21 is a screen shot for the “Select Exercise Type” menu.

FIG. 22 is a screen shot for an example user profile.

FIG. 23 is a series of screen shots for the details of a user profile.

FIG. 24 is a series of screen shots for the details of a user's dashboard.

FIG. 25 is a screen shot of a user's blood glucose history including a graph of blood glucose data.

FIG. 26 is a screen shot of a user's exercise type history including a graph of exercise data.

FIG. 27 is a flow of screen shots for a corresponding digital platform demonstrating the “manage algorithms” module.

FIG. 28 is a screen shot from the digital platform showing a first sample embodiment of a user's “User Analytics.”

FIG. 29 is a screen shot from the digital platforming showing a second example embodiment of a user's “User Analytics,” which includes an Exercise, a Respiratory Rate, a blood pressure, and a blood glucose data summary.

FIG. 30 is a graph of the user's blood glucose data, accessible from the blood glucose summary of the “User Analytics” screen.

FIG. 31 is a graph of the user's exercise data, accessible from the exercise summary of the “User Analytics” screen.

FIG. 32 is a graph of the user's intake vs. output data, accessible from the Active Metabolism Today summary of the “User Analytics” screen.

FIG. 33 is a graph of the user's sleep data, accessible from the sleep summary of the “User Analytics” screen.

FIG. 34 is a screen in the digital platform to manage the data entry for Predictive Algorithm I.

FIG. 35 has screen images from the digital platform to manage the data entry for the AIO Prescription Tool and the AIO Performance Tool, respectively from top to bottom.

FIG. 36 is part one of an Excel document showing variable input and calculated outputs for the APP and digital platform.

FIG. 37 is part two of an Excel document showing variable inputs and/or calculated outputs for the APP and digital platform.

FIG. 38 is part three of an Excel document showing variable inputs and/or calculated outputs for the APP and digital platform.

FIG. 39 is part four of an Excel document showing variable inputs and/or calculated outputs for the APP and digital platform.

FIG. 40 is the Excel document part two for the decision ladder for nutrition showing the formulas in the cells.

FIG. 41 is the Excel document part three of FIG. 38 showing the formulas in the cells.

FIG. 42 is the Excel document part four of FIG. 39 showing the formulas in the cells.

### DETAILED DESCRIPTION

The following detailed description will illustrate the general principles of the invention, examples of which are additionally illustrated in the accompanying drawings. In the drawings, like reference numbers indicate identical or functionally similar elements.

Each pending application and granted patent referenced herein below are each incorporated herein by reference in their entirety.

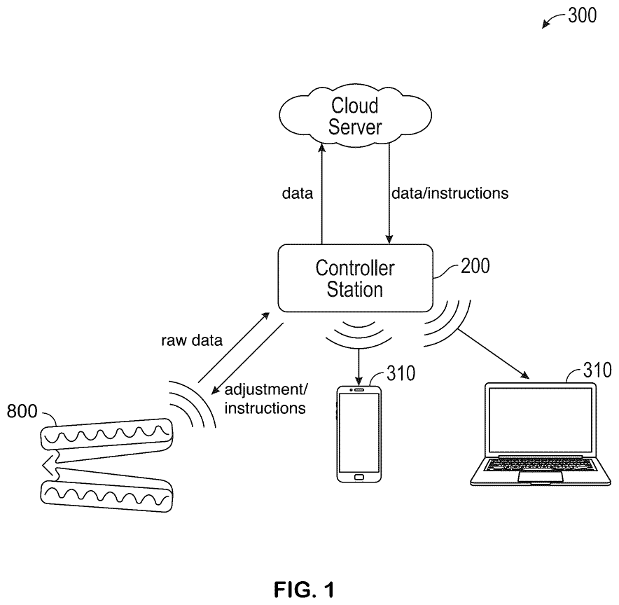

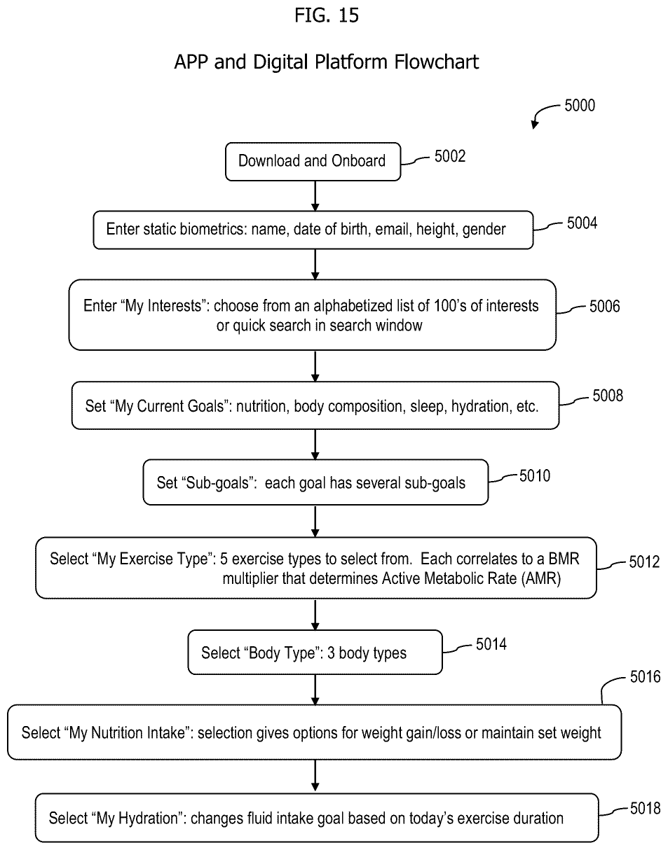

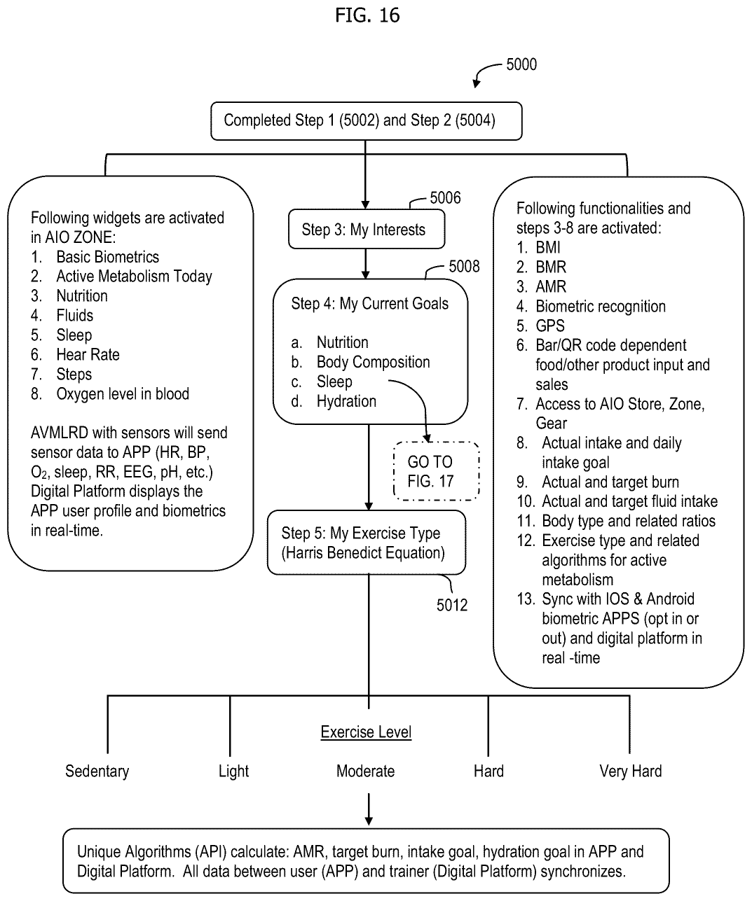

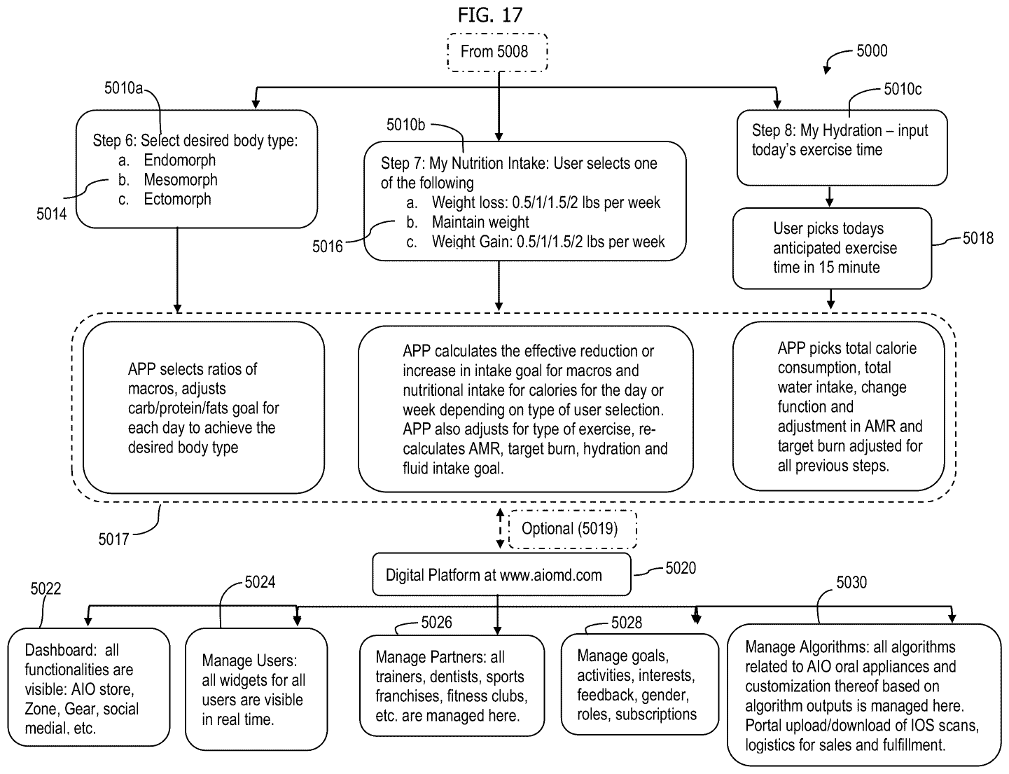

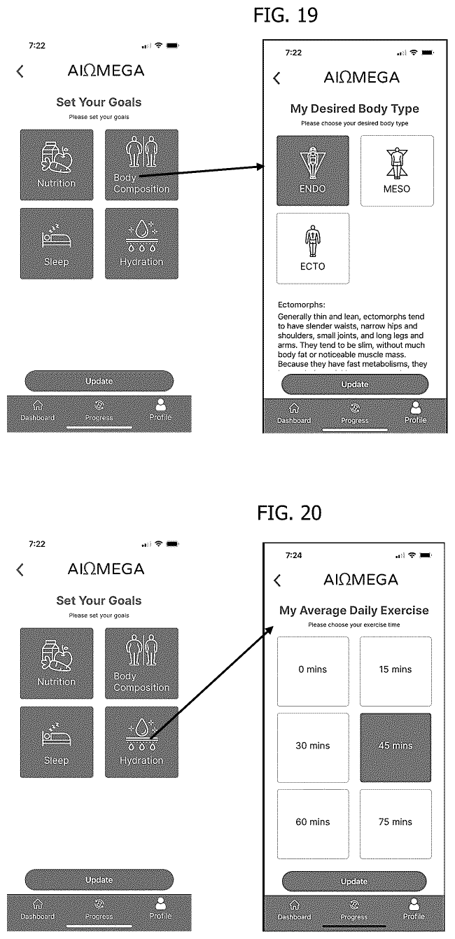

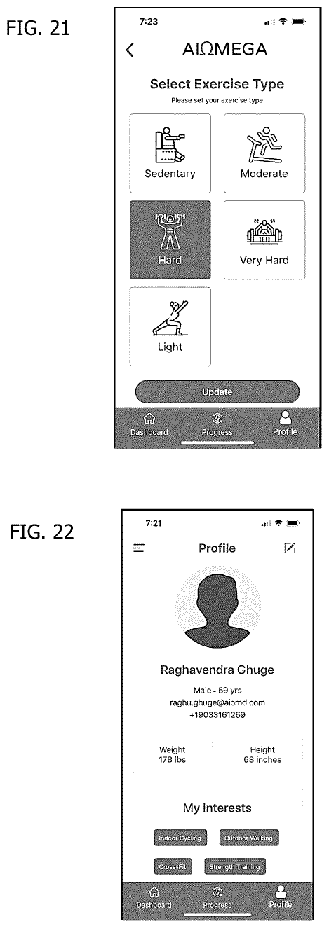

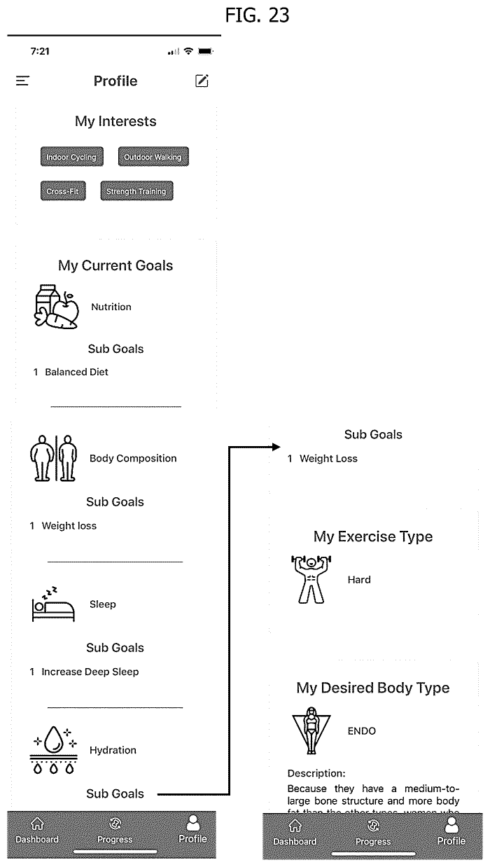

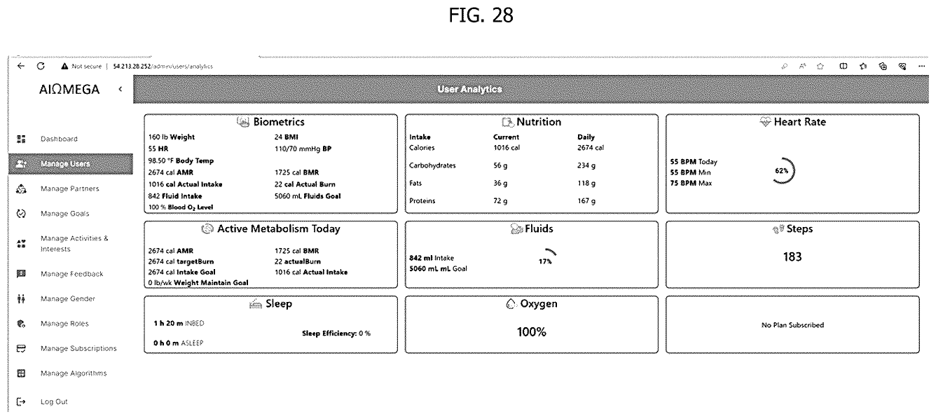

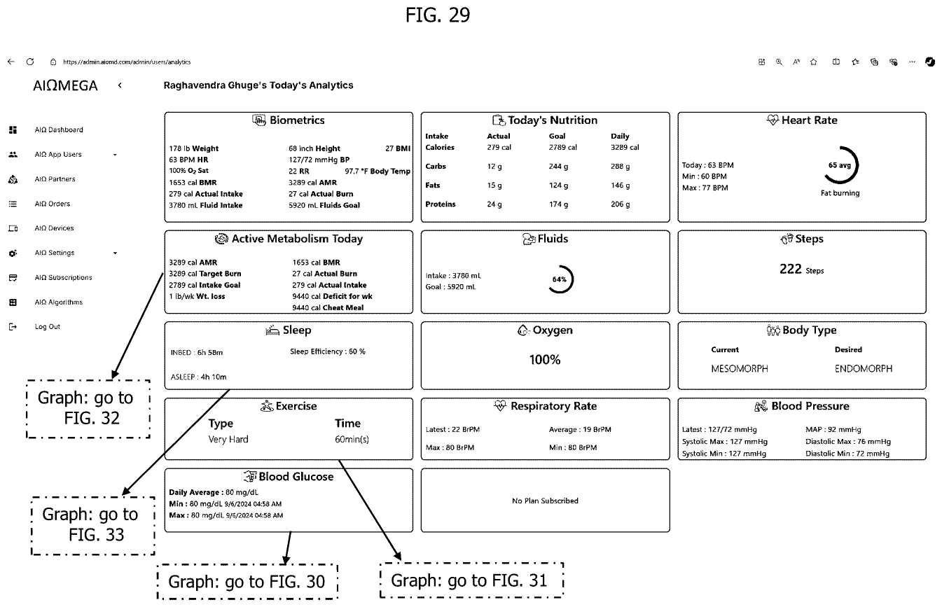

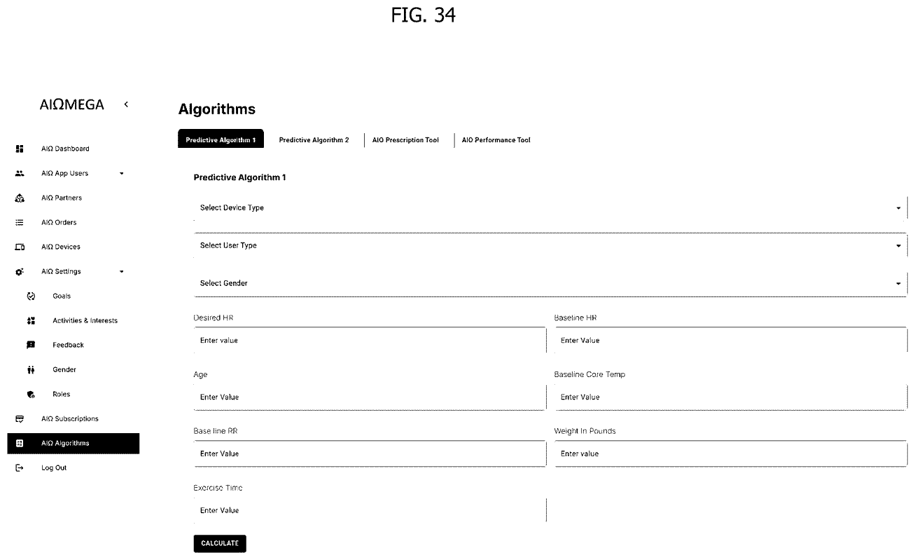

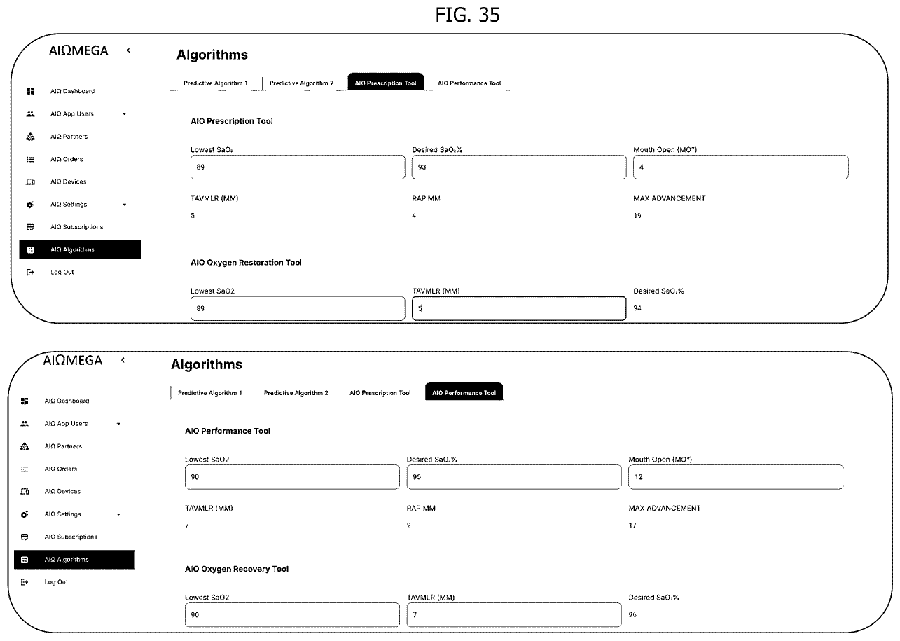

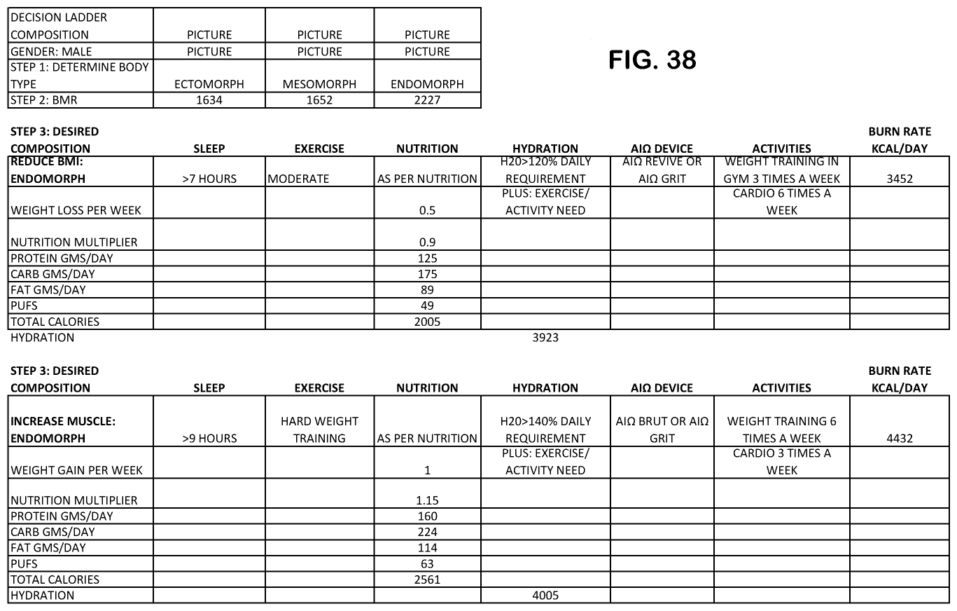

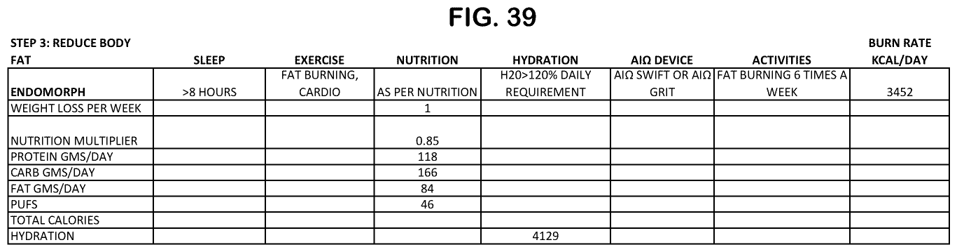

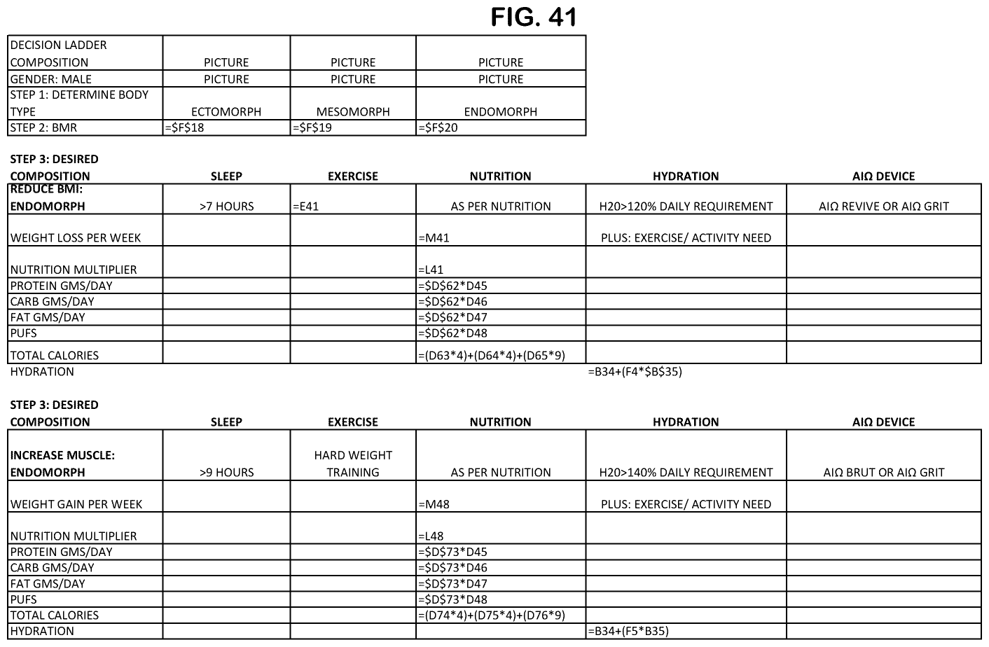

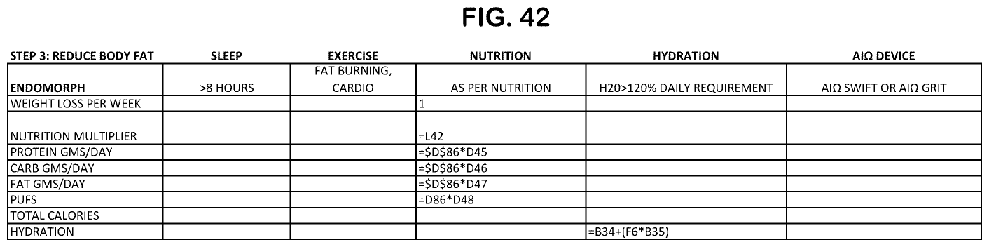

Turning now to FIG. 1, a system 300 is represented that includes a controller station 200 illustrated for operatively controlling any mandibular lingual repositioning devices (MLRDs), generically represented and identified by reference number 800, and communicating with a cloud server, such as those operatively connected via the Internet, and personal electronic computing devices 310. In one embodiment, the controller station 200 can be an independent housing that includes charging capability for the mandibular lingual repositioning devices. Such a controller station 200 is described in more detail in co-pending U.S. Pat. No. 11,484,434. In one embodiment, the controller station 200 may be incorporated into a hand-held smart device and such a smart device would share blue tooth, WIFI, video, audio and communication capability with sensors. The controller station may be miniaturized using PIC/QMC microprocessors for the hand-held device or for a wearable apparatus, such as a wristwatch, wrist band, helmet, waist belt, etc., or may be incorporated into an aircraft or space-ship's internal computing system. The sensors may be on board a MLRD or independent thereof. In one embodiment, the controller can be a proprietary software program for use with an App or can be an App (software application), which are described in detail herein, especially with reference to FIGS. 15 to 42.

System 300 and controller station 200 in all its embodiments will be HIPPA and HITECH compliant for purpose of medical privacy. Interface with the wide variety of electronic health formats (EHR) would allow system 300 and controller station 200 and its operated systems to be available for real-time data download and upload, active health care worker involvement in user's health care needs and would permit the health care worker to operate and alter any treatment and access and interpret diagnostic information provided by the system. As such controller station 200 and system 300 would allow newer formats of health care provisions such as tele-medicine and others yet to be defined. System 300 may be integrated into a full-function health care software-hardware system for patient assessments (such as telemedicine), tests, treatments and medications.

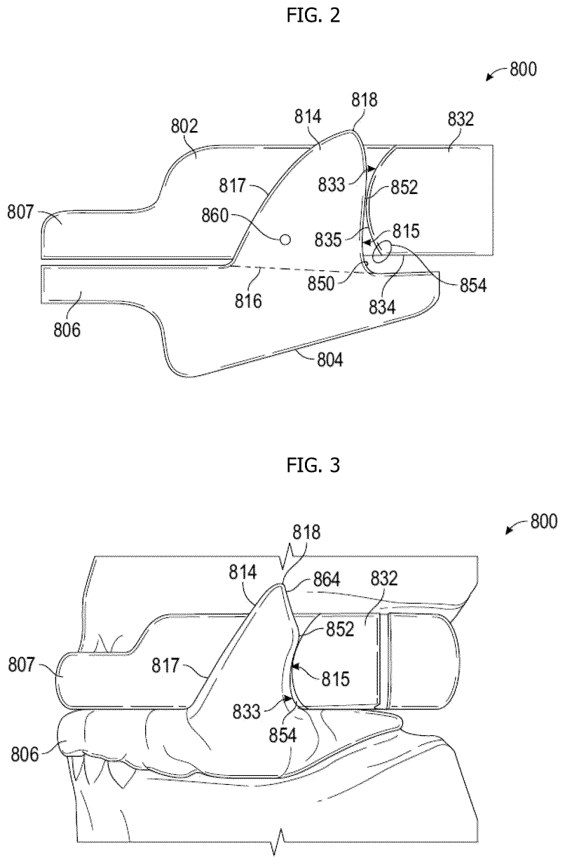

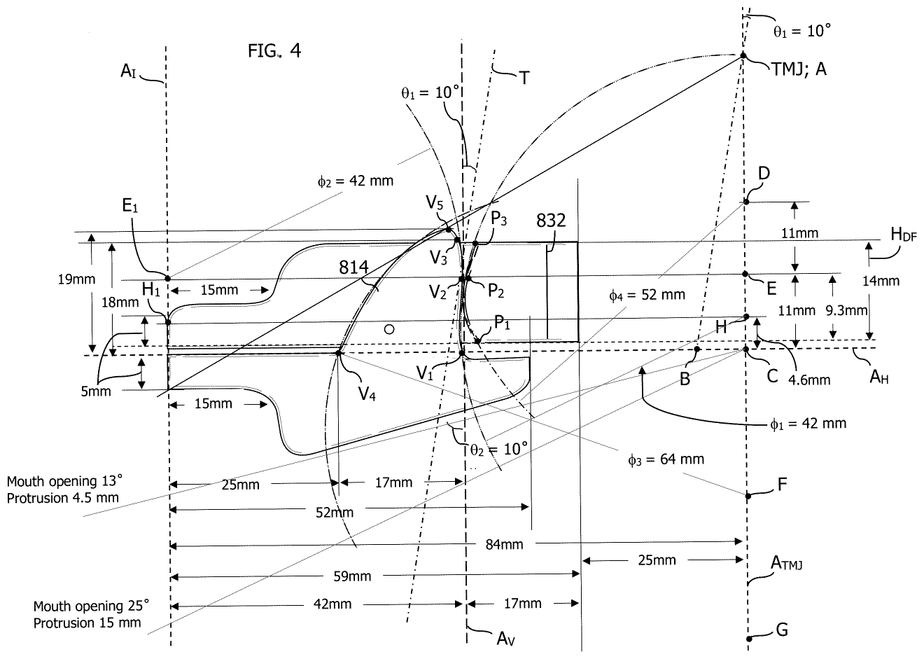

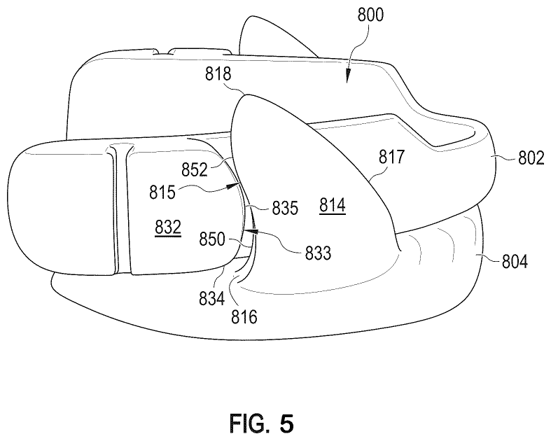

Turning now to FIGS. 2-6, an oral appliance 800 having Dynamic Continuous Open Airway Technology (DCOAT) is shown. The DCOAT is provided by the protrusive flange 814 and driver flange 832 shapes and mating surfaces, which causes the mandible to follow an arc path as shown in FIG. 4 and explained in more detail in U.S. application Ser. No. 17/098,355. As the mandible drops to open the mouth, the mandible will move forward in small increments because of the shape of the protrusive flange 814 and the driver flange 832, thereby opening the airway. These oral appliances are applicable to sleep as well as daytime (awake) related activities, including sports or athletic activities, for all embodiments disclosed herein. The arc demonstrates that when the mandible opens in 5 degree increments relative to the TMJ, the forward point of the mandible changes as shown in Table 1 below.

TABLE 1
Arc₂ Degrees of Travel corelated to Mandible position

| | Distance from the TMJ |
|---|---|
| Degrees of Mouth Opening | (centimeters) |
| 0 | 7 ⅞ |
| 5 | 8 ⅛ |
| 10 | 8 ⅜ |
| 15 | 8 7/16 |
| 20 | 8 9/16 |
| 25 | 8 ¾ |

The oral appliance 800 has a concave-to-convex curvature moving from the base 816 to the most cranial point 818 of the posterior side 815 (or trailing edge) of the protrusive flange 814 of the mandibular piece 804 and a convex curvature 835 of the anterior side 833 (leading edge) of the driver flange 832. While FIG. 2 only shows the left side, it is understood that the right side can be the same. The protrusive flange 814 extends cranially from the mandibular piece 804 which has a teeth covering 806 for the lower teeth. The driver flange 832 protrudes laterally outward from the side of the maxillary piece 802 a distance sufficient to engage the posterior side 815 of the protrusive flange 814 with the anterior side 833 thereof. The driver flange 832 has a base 834 positioned on the maxillary piece 802, i.e., the base of the driver flange does not extend caudally in an overlapping manner with the mandibular piece 804 in this embodiment. However, in another embodiment it may extend caudally as an extension of 835 in a way that it will be alongside (buccal side) of the mandibular piece 804, thus providing additional caudal anterior surface 833 for maintaining articulation with the posterior (trailing) surface 815 of the protrusive flange 814 during extremes of mouth opening such as yawning or otherwise. The maxillary piece 802 has a teeth covering 807 for the upper teeth. The protrusive flange 814 and the driver flange 832 are not shown in these embodiments to have the housings with the motor and mechanism for moving the flanges to provide the movements described herein for the other embodiment, but they are equally usable with such mechanisms and all the systems described in co-owned U.S. Pat. No. 11,806,273.

The teeth coverings of the mandibular piece and or the maxillary piece may be partial (2-5 teeth) coverings covering 2-5 teeth for each of the right and left half thereof or may extend further anteriorly to cover more teeth. The number of teeth covered may be different for the right and left side of each of these pieces as well. Thus, there may be up to 4 individual pieces, two (mandibular and maxillary) for the right and two for the left. This will provide therapeutics for individuals who have a mouth that is too small (micrognathia) to fit an entire bulky device. For most user's the left side and the right side will be mirror images, but if the user has a difference in jaw and/or facial structure making one side different from the other, the device can be custom shaped to accommodate the differences. In any and all of the embodiments disclosed herein, the protrusive flange 814 may be molded as an integral portion of the mandibular piece 804 but is preferably a removably attachable flange. Likewise, the drive flange 832 may be molded as an integral portion of the maxillary piece 802 or it may be removable attachable thereto. The moldable material may be any of those commercially available or hereinafter developed for use in a human oral cavity.

The concave-to-convex curvature of the posterior side 815 of the protrusive flange 814 has a concave portion 850 most proximate the base of the protrusive flange. Cranially above the concave portion 850 is the convex section 852. The shape and positions of the concave and convex portions 850, 852 is described in more detail with reference to FIG. 4. The mathematical model in FIG. 4, was created using a scale of 1 cm=10 mm. Here, the dental horizontal axis (A_{H}) is represented by segment BC and runs horizontally between the mandibular teeth (crowns of the teeth) along the plane and the maxillary teeth (crowns) above the plane. Thus, the mandibular covering's part of the MRD lies below the horizontal axis while the maxillary covering's part lies above the horizontal axis. A vertical axis (A_{V}) is drawn perpendicular to the dental horizontal axis at a position passing between the protrusive flange 814 and the driver flange 832 in the at rest position shown in FIG. 4. The rest position is a position of the mandible at which there is no stress on the TMJ. This axis passes between the mating point V₂ of the protrusive flange 814 and the point P₂ of the driver flange 832. Point A represents the TMJ at rest and an axis parallel to the vertical axis (A_{V}) is drawn through point A, called the TMJ axis (A_{TMJ}). Point B is the point where the horizontal axis and the angle of the mandible intersect. The angle ABC created thus represents an angle adjacent to the angle of the ramus of the mandible. It is typically 40 degrees since the angle of the ramus of the mandible is 140 degrees on average. However, significant age, race and gender variations exist. We have assumed an angle of the ramus to be 100 degrees and the adjacent angle to be 80 degrees for sake of simplicity. The more obtuse the angle of the ramus, the more obtuse the tangent T and the greater the lean in the surface 835 of the protrusive flange 814 and surface 833 of the driver flange 832. Point C is the point where the TMJH vertical axis and the horizontal axis intersect. Point D is a mid-point of the length of the segment AC. Point E is a point along the TMJ axis that is at ⅔ of the height (H_{DF}) of the driver flange 832. Point F is the mirror of point D along the TMJ axis and Point G is the mirror of point A along the TMJ axis, i.e., a negative value equal to point D and Point A, respectively, below the horizontal dental axis. Point E1 is the mirror of point E on a vertical axis parallel to the TMJ axis but positioned at the front of the incisors (A_{I}). Average dimensions were used in FIG. 4, and it is therefore understood that these dimensions may vary from individual to individual based on natural variations of body size, jaw size, head size and variations created by abnormalities of the human body as well.

The primary concept is to use a tangent (T) that is parallel to the lean of the Ramus of the mandible (represented by line segment AB) in relationship to the horizontal axis (A_{H}) that passes between the protrusive flange 814 and the driver flange 832 in the at rest position shown in FIG. 4. This creates an angle within the range of 10° to 50° with the vertical axis (A_{V}) on the maxillary side of the horizontal axis (A_{H}), which we call q₁. For the purpose of the following description and simplicity, 10° was selected for q₁ and q₁=q₂. However, q₁ can be any value within the 10° to 50° range. The tangent (T) defines the point V₂ of the protrusive flange 814 and the point P₂ of the driver flange 832 on the convex portions thereof, which are aligned in the at rest position. This is referred to as point V₂P₂ and is a point where three tangents meet to create the tangent (T). These are designed to meet at the same point although they do not always have to, especially, if a design for any individual requires a variance from this concept. Also, if the Ramus angle is different in each subject from what we have used for this discussion, T may change.

The five points labeled in FIG. 4 for the protrusive flange 814 are identified in this paragraph. Point V₁ is the lowest point on the trailing edge 815 of the protrusive flange 414 where it lands on the mandibular covering of the MRD. Point V₂ is where the tangent (T) coincides with point P₂. Point V₃ is the most cranial point of the trailing edge 815 of the protrusive flange 814. Point V₄ is the lowest point of the leading edge 817 of the protrusive flange 814. Point V₅ is the high point where V₃ reflects and meets the leading edge 817.

The three points labeled in FIG. 4 for the driver flange 832 are identified in this paragraph. Point P₁ is the lowest point of the leading edge 833 of the drive flange 832. P₂ is the point where tangent (T) coincides with point V₂. Point P₃ is the most cranial point of the leading edge 833.

At T=10°, the very front of the incisor part of the MRD to point C (the perpendicular dropped from A) appears to be 84 mm long. The midpoint of this segment is 42 mm (referred to herein as the midpoint length) from either end is at point V₂. This is an average distance and may vary on a case-by-case basis (as will all other measurements). About 4.6 mm above point C is a point that is one third of the height of segment AC measured from the horizontal dental plane, designated as point H. Using point H as a center point, a first arc V₁-V₂P₂ defining the curvature of the concave portion 850 of the trailing edge of protrusive flange 814 is drawn and a second arc P₁-P₂-P₃ (the entire leading edge of protrusive drive 832 is drawn using a radius 1 (Ø₁) of 42 mm (equal to the midpoint length). The 42 mm length for the radius could vary on a case-by-case basis.

The second arc P₁-P₂-P₃ defines almost the entire leading edge of the driver flange 832. The radius that will be used to draw the leading edge of the driver flange is about 0.2-0.5 mm shorter than the radius used to draw the trailing edge 815 of the protrusive flange 814 to allow a small play for the purpose of proper articulation. The leading edge 833 of the driver flange 832 has a back-cut portion 854 most proximate the point P₁. P₁ is described by a different radius, radius 4 (Ø₄) of 52 mm on average. The center point used to draw the arc for the back-cut portion 854 is point D such that segment EC=ED=11 mm.

Point E is created by drawing a horizontal line from the point V₂P₂ such that the angle created by V₂P₂-C-V₁=10° thus allowing the point V₂P₂ to be the point where the tangent T=10° from the vertical axis. Now extending the horizontal line that passes through the points V₂P₂ and E further to the left allows creation of a point E₁, such that segment V₂P₂-E₁=42 mm=segment V₂P₂-E. Extending the line H similarly will allow the creation of H₁. With E₁ as center point using the same radius Ø₂=42 mm another arch is drawn that starts at V₂P₂ and extends upwards to V₃, thus completing the remainder of the trailing edge of the protrusive flange 814. H₁ may similarly be used and any point between E₁ and H₁ may also be used for the same purpose depending on the amount of convexity required at the top of the protrusive flange 814 to create best mandibular advancement for each individual person.

To build the leading edge 817 of the protrusive flange 814, Point F was used as the center to draw arc V₄-V₅. This was then smoothed out at the top for a smooth transition to the trailing edge 815 and to avoid creating pointed edges. The convex curvature of the leading edge 817 is oriented with its curvature tilted toward the TMJ such that the most cranial point 818 (point V₅) is more proximate point V₂ than point V₄. However, turning now to FIG. 3, an alternate embodiment 800 for the MRD is shown in which the leading edge 817 of the protrusive flange 814 can be more linear, yet still oriented tilted with the most cranial point 818 pointed toward the TMJ. Additionally, FIG. 3 has a back-cut portion 864 to the convex portion 852 most proximate the most cranial point 818, back-cut toward the most cranial point 818.

A user in need of an open airway inserts the maxillary and mandibular device of any of the embodiments disclosed herein into their mouth and goes about with their activity or goes to sleep. With respect to the shape of the flanges in FIGS. 2-6, when the user moves the mandible downward, the protrusive flange 814 of the mandibular piece 804 moves along the convex curvature of the driver flange 832, which will move the mandible forward, see the increments of movement set forth in Table 1 above, and naturally opens the airway.

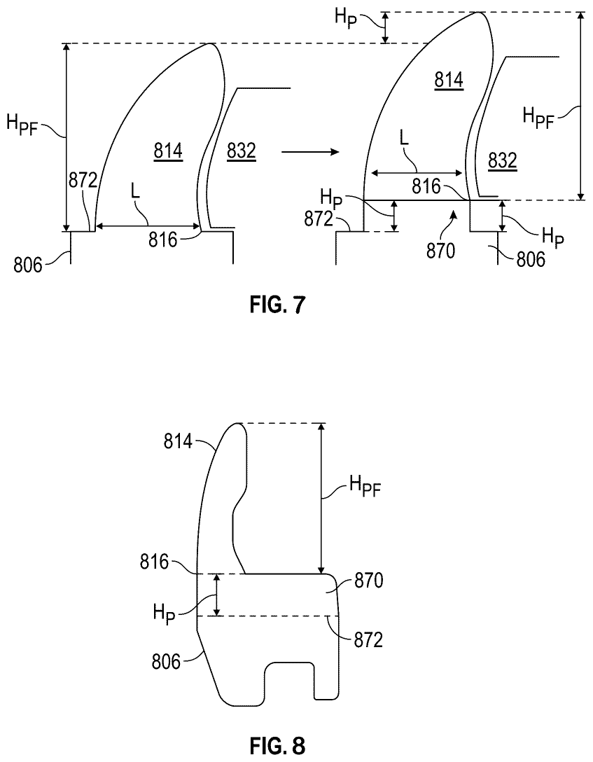

Referring now to FIGS. 7-11, some users are capable of opening their mouths wider than others, and if, their mouth can open to a distance that is greater than the height of the protrusive flange 814 on the mandibular piece 804, there is a chance that the protrusive flange 814 could become disengaged from the driver flange 832 of the maxillary piece 802. To maintain contact between the mandibular piece 904 and maxillary piece 802 (prevent disengagement) during anterior-posterior repositioning and cranial-caudal repositioning, especially when both types of repositioning occur simultaneously, a plateau 870 of a preselected height (H_{P}) has been added to the mandibular repositioning devices between the base 816 of the protrusive flange 814 and the floor 872 of the tooth covering 806 of the mandibular piece. The preselected height (H_{P}) of the plateau 870 prevents disconnect between each protrusive flange 814 and its respective driver flange 832 relative to a fully open mouth measurement between incisors of the user by having a height that compensates for the difference between the fully open mount measurement and the height of the protrusive flange (H_{PF}).

Referring to FIGS. 7-11, the preselected height (H_{P}) for these examples was selected to be 1.06 mm because the user was capable of opening their mount 20.06 mm and the flange height (H_{PF}) was 19 mm. The flange height is typically less than 20 mm and is often in the range of 17 mm to 19 mm for the average adult. In another example, if a user has a fully open mouth measurement of 36 mm and the protrusive flange height is 19 mm, the preselected height for the plateau is 17 mm. As best seen in FIGS. 7 and 8, the plateau 870 extends across the full width of the tooth covering 806 and has a length (L) equivalent to the length of the base of the protrusive flange 814. Here, the plateau 870 has a uniform preselected height (H_{P}) along the length (L) thereof.

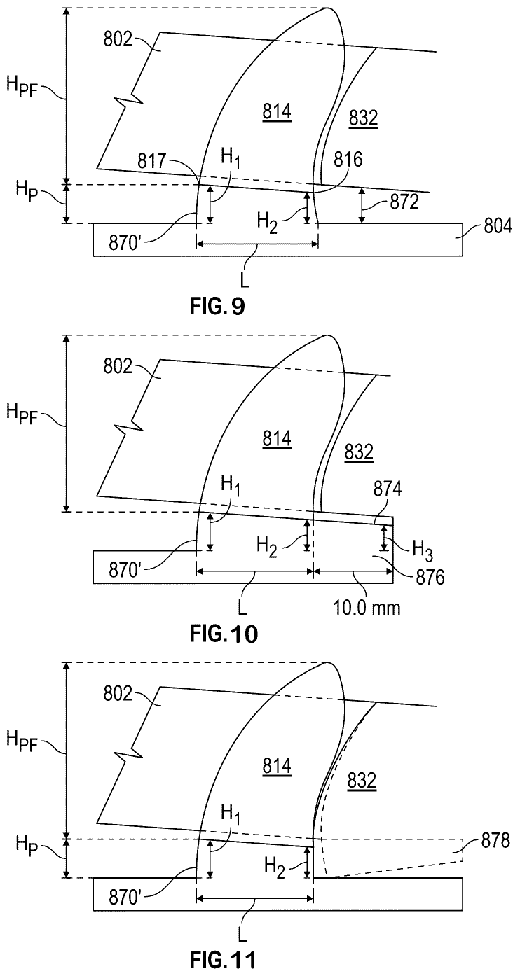

Turning now to FIG. 9, the plateau 870′ is wedge-shaped, thereby having a first height (H₁) at an anterior base 817 of the protrusive flange 814 that is greater than a second height (H₂) at the posterior base 816 of the protrusive flange. The plateau 870 causes the concave-convex curvature of the protrusive flange 814 to be inclined relative to the mandibular piece 804, thus the driver flange 832 is also inclined equivalently to the plateau 870′ to maintain the engaged convex to convex mating curvature thereof. This creates a gap 872 between the driver flange 832 and the mandibular piece 804.

FIGS. 10 and 11 are examples of different embodiments for filling this gap with either an extension of the plateau 870′ or of the driver flange 832, respectively. In FIG. 10, the plateau 870′ has an extension 874 that extends posteriorly to a posterior terminus surface 876 of the tooth covering 806 of the mandibular piece 804. The extension 874 continues the wedge shape of the plateau 870′ and terminates with a third height (H₃) that is less than the first height (H₁) and the second height (H₂). In FIG. 11, the plateau terminates posteriorly at the posterior base 816 of the protrusive flange 814 and the driver flange 832 has an extension 878 that extends caudally with its anterior side extending the convex curvature thereof past the posterior base 816 of the protrusive flange 814 into a gap 872 (FIG. 9). The driver flange 832 has a base that is seated on the mandibular piece 804 as a result of the presence of the extension 878. The extension 874 and 878 provide the benefit of added space for the housing in which the electrical components, motors, power sources, etc. that are described herein are enclosed. In another embodiment, extension 878 may extend caudally adjacent to the mandibular piece only thus allowing an extension for continued articulation between surfaces 833 and 815 with extremes of mouth opening. The mathematics and benefits of the plateau feature is further described in U.S. Pat. No. 11,806,273.

Beside a plateau adjustment, the protrusive flange and driver flange can be adjusted with respect to its respective posterior lean. Posterior lean is described in more detail in U.S. Pat. No. 11,806,273. The convex curvature of the driver flange can be increased, decreased, or adjusted with a preselected amount of posterior lean. Such changes to the convex curvature are selected to enable the mandible of the user to advance incrementally more with mouth opening. Less superior lean advances the mandible in the anterior direction less with each degree of mouth opening. In contrast, more superior lean advances the mandible in the anterior direction more with each degree of mouth opening. As noted above, the pairs of flanges are typically mirror images of one another unless the user has a jaw or face asymmetry.

As disclosed in U.S. Pat. Nos. 11,484,434 and 11,806,273, the oral appliance can include motorized and/or robotic drivers integrated into the maxillary piece 802 and mandibular piece 804 to affect anterior-posterior movement and cranial-caudal movement of the mandibular repositioning device, can include sensors, can include a lingual flange, electrode stimulators, medical dispensers (configured to dispense powders, pellets, tablet, liquids, or aerosolizable medicines), etc., and combinations thereof. The sensors are typically in the oral cavity but are not limited thereto. The sensors can measure airway cross-sectional area, airflow volume, airflow velocity and pressure, airflow resistance, systolic and diastolic blood pressure, electrical activity of the heart, oxygen level, heart rate, and combinations thereof. The systolic and diastolic blood pressure and electrical activity of the heart may be measured by capacitive micro-machined ultrasound. Position sensors can be included that measure a first distance for cranial-caudal movement and a second distance for anterior-posterior movement of the driver flange. The sensors can include but are not limited to a pulse oximetry sensor, a vibration sensor, an airflow sensor, a pH sensor, an EKG sensor, electro-encephalogram (EEG) sensor, electromyogram (EMG) sensor, electro-oculogram (EOG) sensor, lactic acid sensor, a pulse transit time (PTT) sensor, an ultrasound sensor (echocardiography), a doppler ultrasound, an M-mode ultrasound, a 2D ultrasound, a 3D ultrasound, a pressure plate, a temperature sensor, a body position or jaw position sensor (such as a potentiometer), glucose sensor (including a blood glucose level in the tongue or soft palate), CMUT/IVUS doppler ultrasound, nerve conduction (NC) data from the nerves of the tongue, pharynx and muscles of mastication (jaw muscles) and phonation (speech), CDT/CNT based infra-red oxygenation receptors, heart rate, a pressure measurement sensor, a hygrometer sensor, respiratory rate sensor, core body temperature sensor, a microphone or sound recording sensor, non-invasive ventilation systolic/diastolic blood pressure sensor, a carotid doppler (trans-oral) sensor, temperature and humidity derived from respiratory (inspiratory and expiratory) airflow, computational mini-Incentive Spirometry based on above inspiratory-expiratory airflow or time ratio (early detection of exercise-induced asthma in an athlete, a soldier or a fitness or weight loss buff), a cardiac trans-oral echocardiography sensor, video recording, sound recording, and hygroscopic/hydration sensor. Any number of combinations of the sensors listed above is possible and can best be selected by a medical professional based on data relative to the pre-selected end user. Sensors in the left side and right side could be symmetric or complimentary or asymmetric. The EKG sensor may have better reading from the right side than from the left side and thus is placed on the right side. Together, the EKG sensor and ultrasound sensor create complete cardiovascular hemodynamic data. The sensors will provide data and feedback to controller 200 for multiple purposes including allowing the controller to make fine adjustments to all components of the system. The data interfaces with standard Bluetooth functionality or WIFI functionality and the controller station may be used as a mobile unit with Bluetooth and WIFI functionality and as such may be carried to work or elsewhere since it has its own rechargeable battery operations. Controller station will be interfaced with proprietary or open platform program that can be securely loaded on variety of computer systems and hand-held smart devices.

The system 300 can create three-dimensional images and videos of breathing, cardiac function, carotid blood flow data, eye-movements, jaw movements and brain EEG recordings for identification of medical conditions and interventions that may be useful to correct or treat medical conditions.

When the electrode stimulator is present it can be housed within a lingual flange that extends from the mandibular piece, as described in U.S. Pat. No. 11,484,434, to lie under the tongue in contact with lingual muscles, in particular the Genioglossus (GG), the Geniohyoid (GH), sub-mentalis (SM), and Glossopharyngeal (GP). The lingual flange can also house therein, in a fluid-tight manner, one or more of the sensors and the necessary hardware and power to operate the electrode and the sensors. In other embodiments, the electrode stimulator can be a lateral pterygoid stimulator, medial pterygoid, or masseter stimulator.

In one embodiment, the medicament is radiation pellets for treatment of oral cancer or immuno-therapy. In another embodiment, the medicament is trans-mucosal or sublingual drugs, for example, but not limited to, nitroglycerine, intermezzo, albuterol, ADVAIR® medicine. In an embodiment where the medicament is intermezzo, the sensor is an EEF, EOG, or EMG sensor to detect insomnia and thereafter dispense the intermezzo. In another embodiment, the medicament is nitroglycerine and the sensor is an EKG monitor. Additional sensors are beneficial with this embodiment, including a blood pressure sensor, echocardiography and/or carotid doppler blood flow. In a third embodiment, the medicament is a dry powder micro-aerosol inhalation of insulin to treat diabetes and the sensor is a non-invasive continuous glucose sensor. In a fourth embodiment, the medicament is a bronchodilator and the sensor is a microphone to detect breathing difficulties such as wheezing, for example in asthmatics.

In one embodiment, the medicament is in pellet form and the pellet is filled with a liquid or aerosolized form under pressure therein. The pellet is rupturable, meltable, pierceable, or dissolvable A rupturable pellet ruptures upon application of pressure, such as being squeezed by a driver of a piezo electric motor. A meltable pellet open upon application of heat, such as heat from the power source via a heating electrode. The pellet may have a predesignated location that is made of meltable material or dissolvable material which upon disintegration releases the said medication or be an on-off robotically operated valve that opens to release pre-determined concentration or dose of medication and then shuts off. A pierceable pellet is opened by a micro-needle within housing. A dissolvable pellet is/could be simply ejected into the oral cavity and dissolves upon contact with saliva. Each pellet is a single dose unit of the selected medicament relative to the user.

The system 300, in addition to sensors within or onboard the MLRD 800, can wirelessly communicate with additional sensors connected to the user to provide a broader data set for a more complete picture of the user's physiology. For example, electrocardiogram (EKG), electromyography (EMG), electrooculography (EOG), electroencephalography (EEG) sensors, echocardiography, blood pressure monitoring systems, and sensors sensing environmental conditions, such as temperature, ambient light, and humidity. The system may include a camera for video recording through the controller station 200 to evidence any nocturnal seizures, sleep-walking, other movement or violent disorders during sleep.

Referring again to FIG. 1, the personal computing device 310 likely encloses a circuit board having a microprocessor, including memory (non-transitory computer readable media) in which is stored firmware, has a receiver configured to receive electronic communications, and has a transmitter configured to send electronic communications, including wireless communication capabilities to electronically communicate with at least the controller 200, but can also be configured for direct electrical communication with the MLRD 800 for real-time communications with any onboard sensors.

The firmware and algorithms, including learning algorithms as well as standard algorithms, stored in the memory of the circuit board of the controller station, or in a digital platform of the cloud server or of a personal computing device may define myriad parameters for configuring the protrusive flange and driver flange and the movements of the MLRD 800, which can be done in real-time. When the MLRD 800 includes the motorized components and robotics described in the patents incorporated herein by reference, the airway of the user can be opened without disturbing the sleep of the user, wake related fitness, or any other activity of the user. Algorithms designed to record, interpret, and analyze, execute commands, and facilitate feedback functions will contain tolerance range, critical values, and reportable values. Similar application may be appropriate for sports, athletics, performance, and military users.

Turning now to FIGS. 15-17, a system 5000 is illustrated using a plurality of flowcharts in which a computing application 5002 implementable on a user's personal computing device is configured for operative electronic communication 5019 to and from a managing platform 5020 of a supervising manager. The computing application 5002 (referred to as an “app”) is downloadable software commonly available via an iOS platform or Android platform. The APP 5002 defines a combination of widgets and applications that connect various biometric devices, mobile operating systems, a variety of interfaces such as Bluetooth, NFCs through building SDK's and API's and other methods of connectivity. The APP 5002 is configured to connect to any of the oral appliances or oral devices described herein, such as those that enhance sleep and/or fitness, and to future oral appliances/devices that have sensor technology incorporated therein. The goal is to track, monitor, and intervene in order to produce healthier outcomes in individual users while preventing the onset of various medical and unhealthy outcomes. The user can have one of the oral appliances, but it is not required to use the App. Some of the oral appliances are commercially available under the brand AIO™ SWIFT™, GRIT™, BRUT™, BREATHE™, and REVIVE™ oral appliances.

These oral appliances have a maxillary piece configured to cover teeth of a user that has a driver flange protruding laterally outward on either or both of the right side and left proximate a user's molar teeth, each driver flange has an anterior side with a convex curvature, and have a mandibular piece configured to cover teeth of a user that has a protrusive flange extending cranially therefrom positioned to have a posterior side engaged with the anterior side of each driver flange. The posterior side of each protrusive flange has a concave-to-convex curvature from a base of the protrusive flange toward a most cranial point of the protrusive flange and a convex portion of the concave-to convex curvature engages the convex curvature of the driver flange in a rest position. The interface of the protrusive flange and driver flange are configured such that when the user moves the mandible downward, the protrusive flange of the mandibular piece moves along the convex curvature of the driver flange, which will move the mandible forward incrementally and naturally opens the user's airway. The mandibular piece of the oral appliances can include the plateau of a preselected height, described above, between the base of the protrusive flange and the tooth covering. The preselected height is set to prevent disconnect between each protrusive flange and its respective driver flange relative to a fully open mouth measurement between incisors of the user; thus, preventing the mandible of the user from falling backwards and closing the airway. The system not only advances movement of the mandible (cranially and anteriorly) but enables a relaxed movement of the mandible (caudally and posteriorly), which allows the temporomandibular joint to relax periodically to prevent jaw discomfort, temporomandibular joint strain and destabilization, morning stiffness of said joint, and alteration of the user's bite.

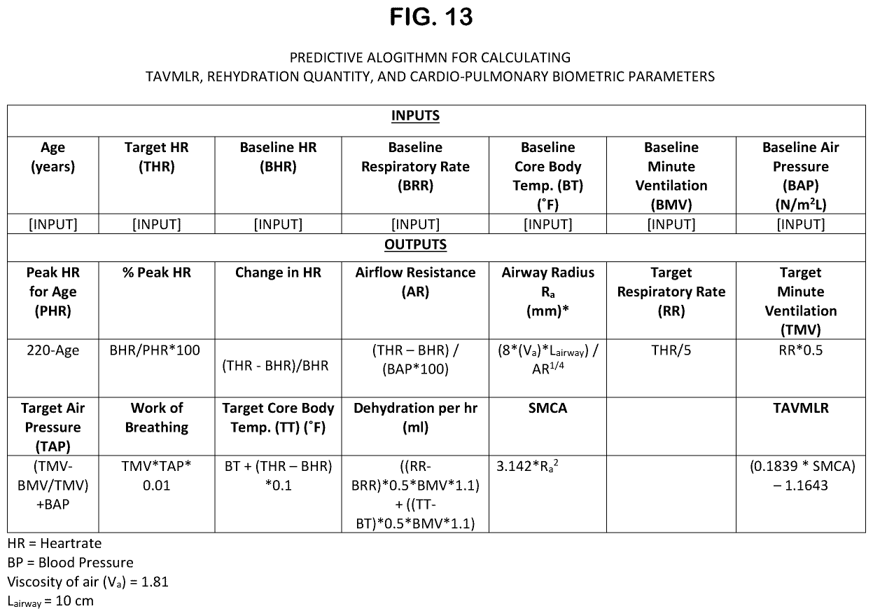

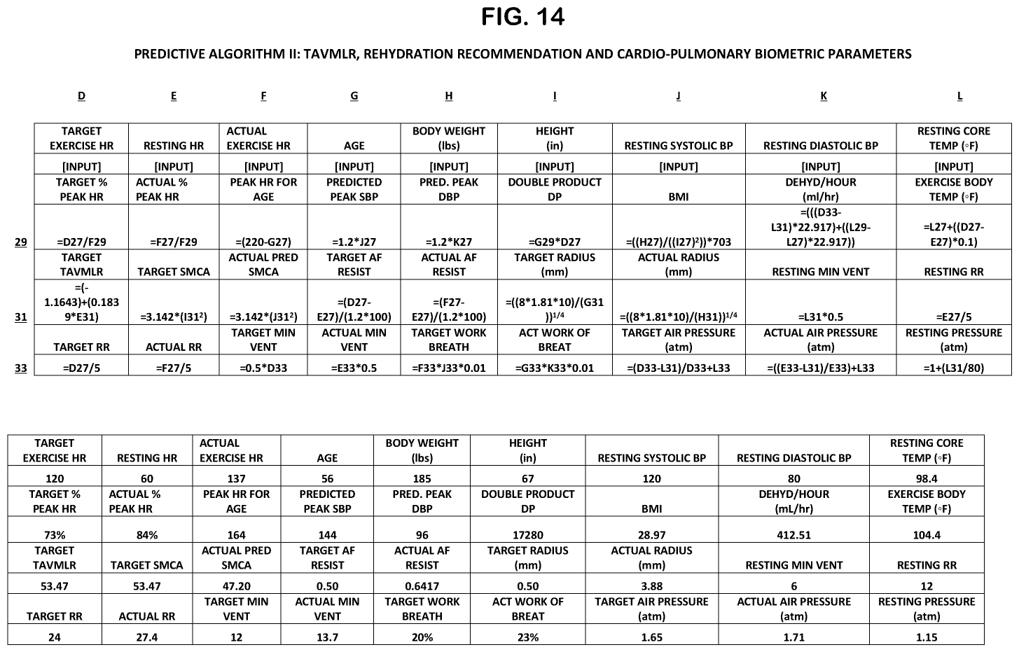

Still referring to FIGS. 15-17, the downloadable software 5002 comprises a plurality of modules and instructions, wherein the plurality of modules includes a first module 5004 configured for entry of a user's biometrics, a second module 5008 configured for entry of a user's health and/or fitness goals, a third module 517 configured for calculating numerous variables relative to the user's health and/or fitness goals, and a fourth module 5019 configured for operable electronic communication with a managing platform of a professional. This fourth module 5019 is configured to be activatable and de-activatable by the user. The numerous variables comprise one or more of carbohydrate, protein and/or fat daily intake, calories per day and/or calories per week, basal metabolic rate, ml/hr hydration loss during exercise, any or all of the variables in FIGS. 13-14 according to the equations therein, including but not limited to water intake, active metabolic rate, and a target calorie burn rate. In another embodiment, the third module configured for calculating the numerous variables can be in the managing platform (digital platform) in addition to the downloadable software or as an alternative to being in the downloadable software.

The first module 5004 collects static biometrics of the user, such as their age, name, email, etc. to build connectivity, push notifications etc. The second module 5008 collects dynamic inputs form the user. These dynamic inputs are primarily in six categories: My interests 5006, My Current goals 5008, My Exercise types 5012, My desired body type 5014, My Nutrition 5016 and My Hydration 5018, which includes Today's Exercise Time, as labeled in FIGS. 15-17, which may each have sub-goal selections 5010a-c thereunder. There is customizability of each of these inputs at the end-user level in the App and also at the managing platform level on the back end of the digital platform. The managing platform can push out new goals, modify, or add new sub-goals for optional section by an end-user or all end users. Analytics assisted tailoring of end-user goals and sub-goals will be made possible through the managing platform.

The App is configured to receive inputs data inputs from (i) blue tooth devices, such as scales, water bottles, watches, Oura ring; (ii) operating systems such as iOS Health Kit, Android Google Fitness, etc.; (iii) other Apps, especially those related to health, nutrition, and/or fitness; (iv) GPS tracked restaurants, groceries, etc.; (v) food container QR codes and barcode; (vi) remote non-invasive or invasive continuous or intermittent glucose monitoring system(s). Some example biometrics that can be a data input received by the App include heart rate, active metabolic rate, sleep data, number of steps per day, body type, fluid intake, respiration rate, oxygen intake, blood oxygen level, blood glucose level, blood lactate level, etc.

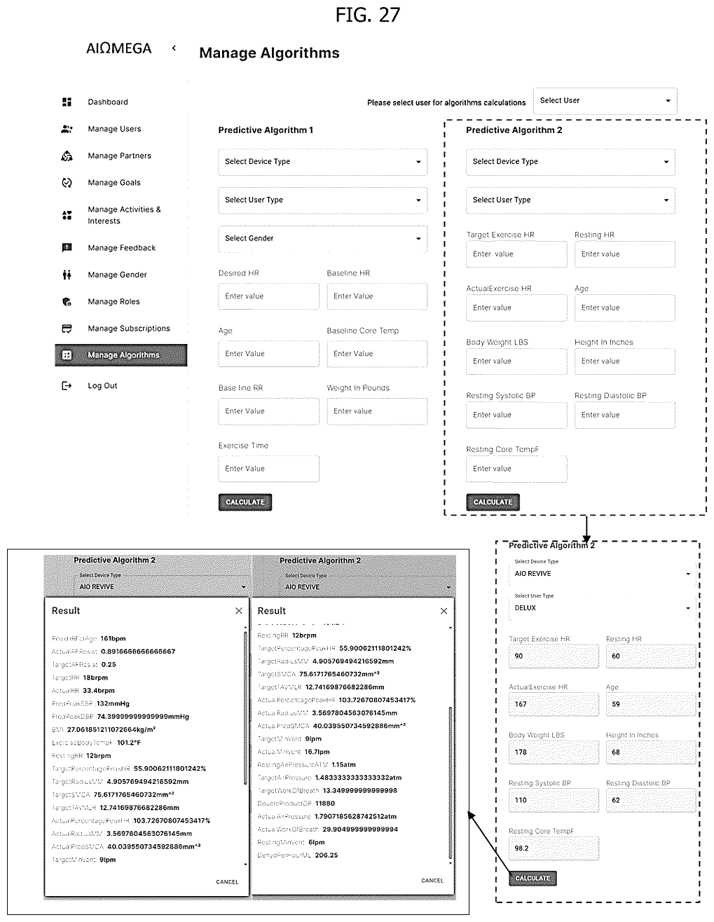

The managing platform 5020 of the professional is configured for operative electronic communication with the computing application 5002 over a communication network, such as the internet or any equivalent thereof. The managing platform 5020 comprises a user interface, see FIGS. 27-29, configured for implementing a plurality of modules 5022, 5024, 5026, 5028, 5030 and instructions. The plurality of modules comprises a fifth module 5024 for managing the data of and from the user's computing application, a sixth module 5026 configured for managing which professionals have access to the user's computing application, a seventh module 5030 configured for managing one or more algorithms executable by the third module of the computing application. The plurality of modules can also include an eight module 5022 configured to display a functional dashboard of options and a ninth module 5028 for managing the goals, activities, interests, feedback, roles, subscriptions, etc. This managing platform 5020 connects the end-user of the computing application with health and fitness professionals through an interface that promotes healthy choices for humans and becomes an easy (all in one place) access point resource for health and fitness products. The health and/or fitness professional can be a fitness trainer, an athletic coach, an athletic trainer, a dentist, a doctor, a nurse, a dietician, a health coach, an athletic director or athletic organizations, weight loss programs, rehabilitation centers, rehabilitation programs, cardio-pulmonary programs, hospitals, nursing homes, fitness equipment manufacturers, nutrition product companies, hydration product companies, and the like. The supporting professionals, via the managing platform, see the information from the end-user's dynamic and trackable data, thus facilitating meaningful decision making and accountability. Onboarding of relevant stakeholders is performed by administrative user to regulate privacy, protect individual consumer information, utilize data to develop normative and abnormal values for populations and groups of consumers such as to generate guidelines for algorithms to perform recommendations or therapeutic interventions controlled by individual stakeholders. AI based algorithms are generated and they provide guidance for precision in decision making and enhancements to outcomes, including forecasting AI algorithms and predictive AI algorithms.

In the systems disclose herein the one or more algorithms and user data cooperate operatively within the third module to iteratively evaluate the variables configuring the protrusive flange and the driver flange of the user, wherein the output of the third module is a recommended change to one or more of the variables of the user's protrusive flange or driver flange. The variables of the protrusive flange and driver flange include one or more of the curvature, height, width, lean, thickness, yaw, rotation, position on the mandible piece or maxillary piece, as well as the position of sensors. The recommended change is set to incrementally increase or decrease a health or fitness parameter of the user. Some example health or fitness parameters include but are not limited to air flow volume, air flow rate, oxygen levels, glucose levels, blood pressure, heart rate, respiratory rate, core body temperature elevation during a physical activity, release of aerosolized medications, inhalation of aerosolized medications, running speed, and blood sugar values.

In the system the supervising manager of the user has access to the one or more algorithms to adjust variables to determine a desired health or fitness parameter or one or more of the variables of the protrusive flange and the driver flange. The supervising manager can be a dentist, a medical professional, a health professional, an athletic trainer, an athletic manager, a weight loss trainer, a physical fitness trainer, an athletic coach, a dietician, and the like. The supervising manager can use the digital platform and the algorithms therein to create or build incremental improvements in the user's oral appliance flange technology. The digital platform can include a plurality of forecasting-AI (F-AI) and predicative-AI (P-AI) that determine improvements in health/fitness outcomes based on the incremental improvements in flange technology.

The supervising manager may be interested in user data related to air flow volume or flowrate, oxygen levels, glucose levels, blood pressure, heart rate, respiratory rate, core body temperature elevation with effort, speed of running, reduction in BP, lowering of blood sugar, and/or many other biometrics including those that evolve as new sensors are invented. The supervising manager may also be interested in delivery of medicaments, including but not limited to release of aerosolized insulin to reduce blood sugar, inhaled breathing medication or heart medication to reduce or increase relevant biometric parameter measured through sensors in the oral appliance or external sensors.

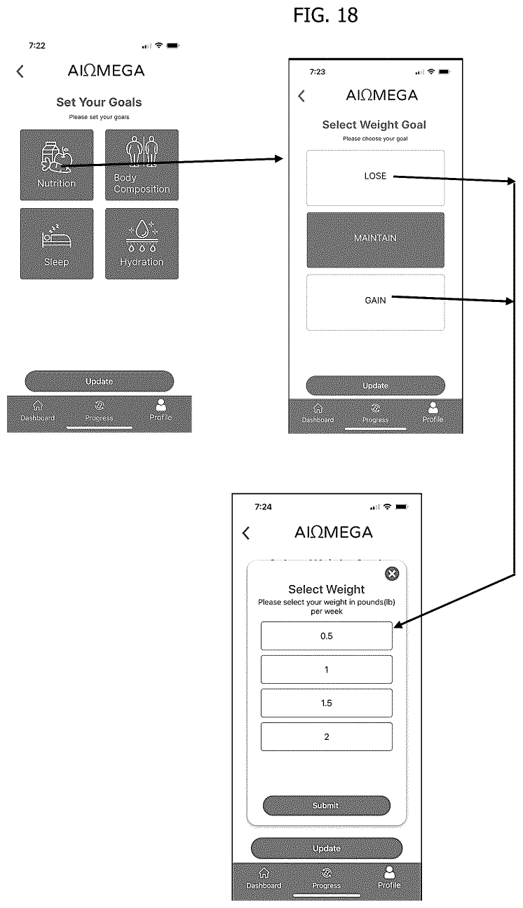

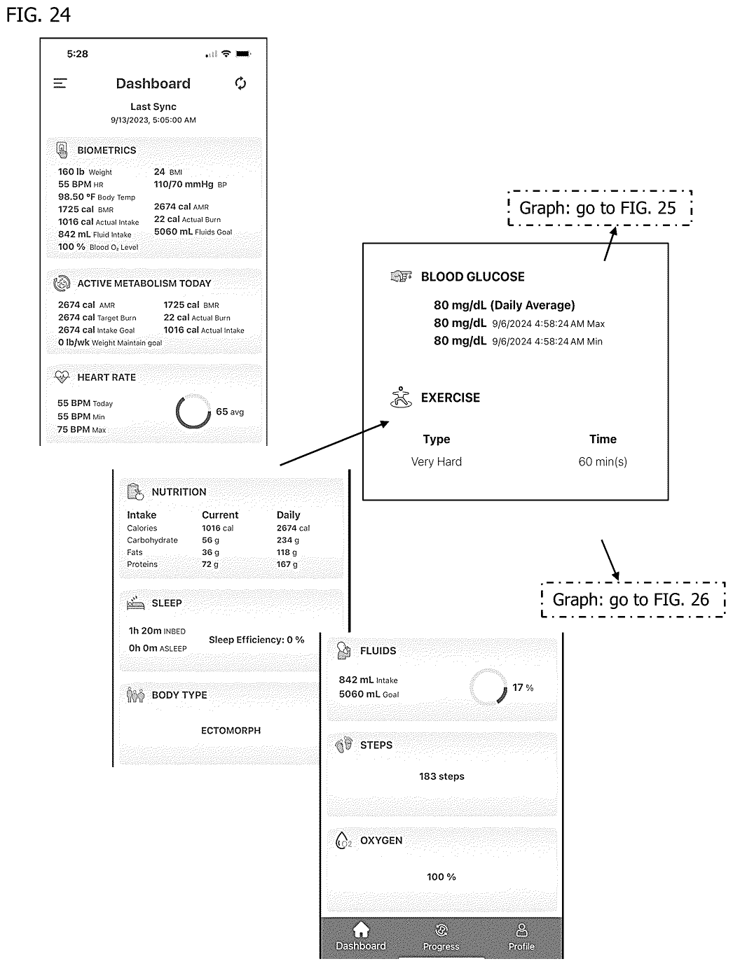

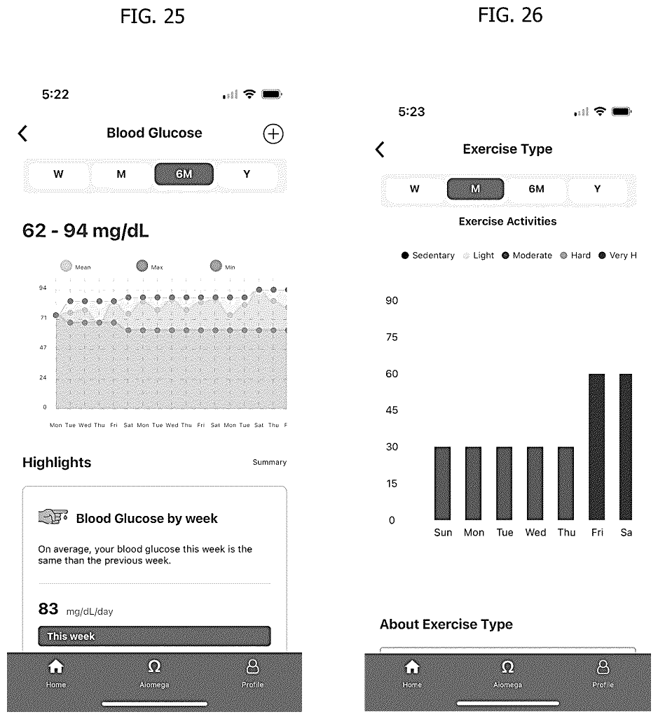

Turning now to FIGS. 18-26, screen shots from the APP are provided showing the various goals an individual can set, the exercise type selection, the users profile, the user's dashboard, and user data in graph form. The dashboard, shown in FIG. 24, includes the user's biometrics, active metabolism today, hear rate, nutrition, sleep data, body type, fluid intake and fluid goal, steps walked, oxygen level, blood glucose, and exercise completed. FIG. 25 is a graph of a user's blood glucose over a 6 month period. There data includes options of graphing data from a week, month, six month, and year. FIG. 26 is a graph of exercise type showing the duration and level of the exercise, selected from sedentary, light, moderate, hard, and very hard.

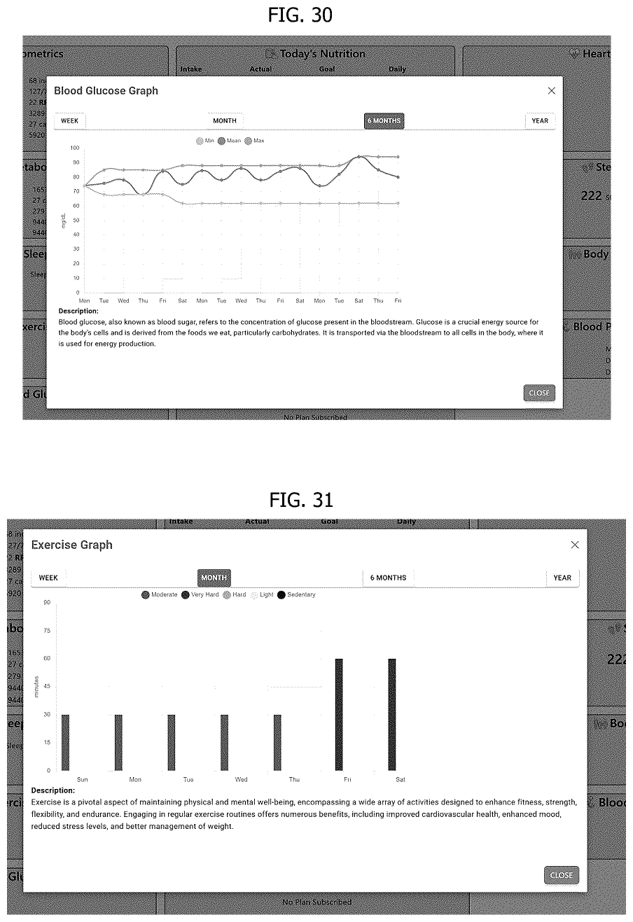

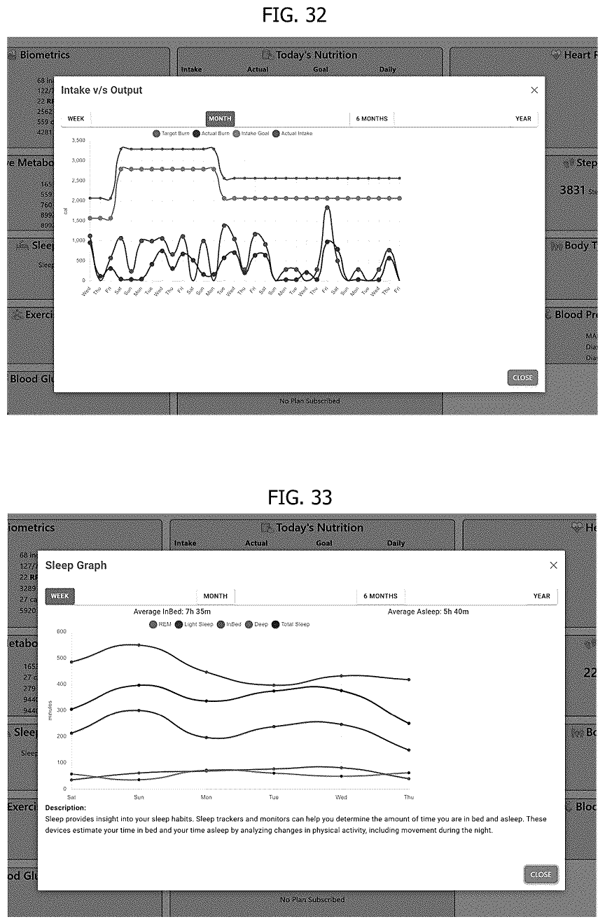

Turning now to FIGS. 27-29, screen shots from the digital platform are shown, which also reflect the data found in the user's dashboard. From this user analytics screen, graphs for blood glucose (FIG. 30), exercise (FIG. 31), active metabolism (FIG. 32), and sleep (FIG. 33), etc. can be accessed.

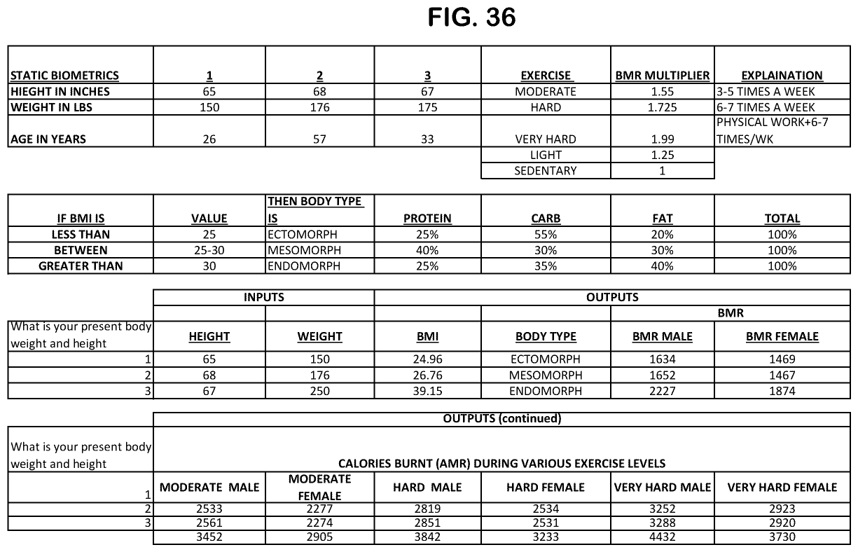

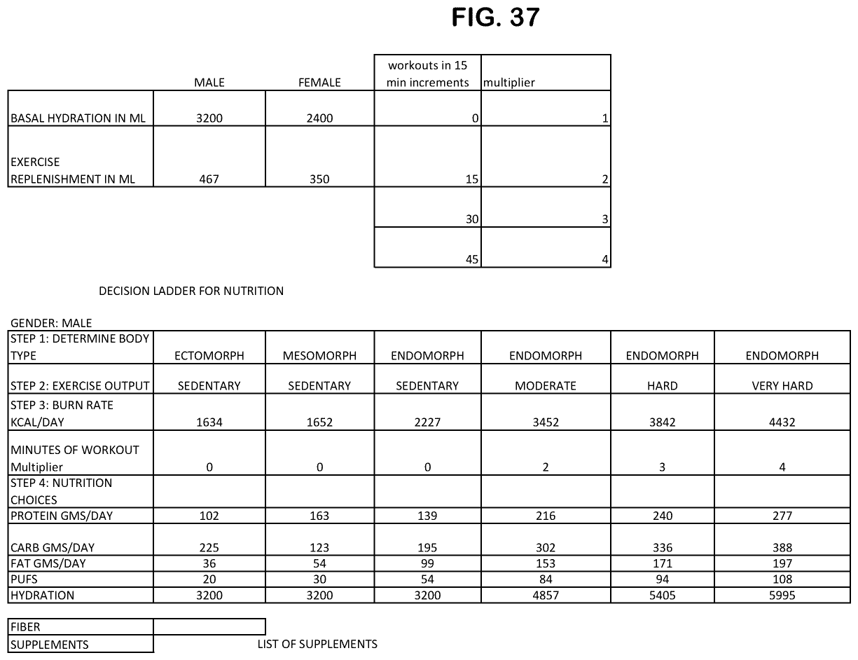

Turning now to FIGS. 27 and 34-35, the digital platform also includes screens from which various algorithms can be managed or implements, adjusted, and new outputs calculated. FIGS. 36 to 42 provide background excel documents representing some of the data and calculations for one or more of the algorithms. With respect to FIGS. 36 and 37, some of the mathematical formulations are set forth below.

$$\begin{matrix}BMI = ( weight / {( height )}^{2} ) * 703 ; \\ {BMR}_{male} = 88.362 + ( 13.397 * ( height / 2.2 ) ) + ( 4.7999 * ( weight * 2.5 ) ) - ( 5.677 * age ) \\ {BMR}_{female} = 447.593 + ( 9.247 * ( height / 2.2 ) ) + ( 3.098 * ( weight * 2.5 ) ) - ( 4.33 * age )\end{matrix}$$

Calories Burnt during exercise is multipler (M)×BMR male or female. The multiplier is: moderate exercise=1.55; hard exercise=1.725; very hard exercise=1.99

$$\begin{matrix}Basal {hydration}_{male} = 40 * ( weight / 2.2 ) and basal {hydration}_{female} = 30 * ( weight / 2.2 ) \\ Exercise replenishment in ml = ( basal hydration / 24 ) / 3.5\end{matrix}$$

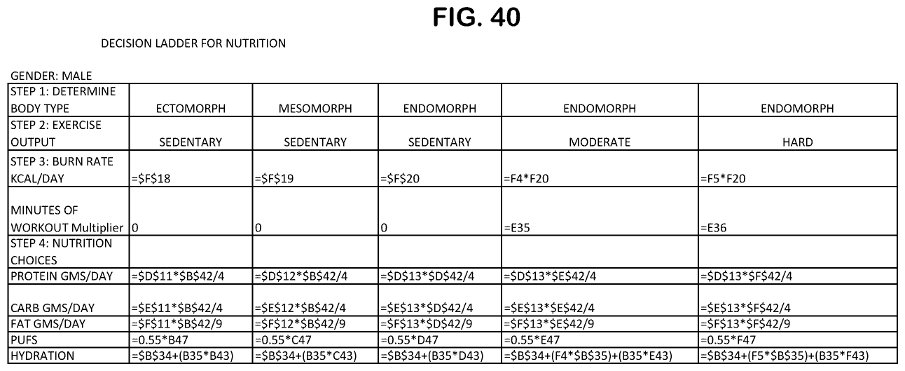

The remainder of the mathematical formulations are set forth in FIGS. 40-42.

The system 300 can also be configured to send data to a pharmacy, emergency medical services or HIPPA validated designated caregiver. The server can also send commands, configuration data, software updates, and the like to the controller station 200. The configuration data may include, but is not limited to, configuration parameters for the system 300, configuration parameters for a particular user, and/or notifications, feedback, instructions, or alerts for the user.

Age and gender specific physiology of the airway and the mouth during sleep are known to affect sleep and cause sleep disorders. The system 300 includes user data that is age and gender specific and algorithms that reflect the same, see FIGS. 36-39, which can improve outcomes. System 300 has ability to create database of all physiological and pathological events measured in real-time and time synchronized with each other in its users and develop algorithms for normal and abnormal manifestations of disease states during wake and sleep and develop new cause-and-effect understanding of these events that have never been observed before. Recording and correlation of these phenomenon with sensors, especially during sleep would help understand conditions such as ‘wake-up strokes’ (occur during sleep) that account for 14% of all strokes and diagnose conditions like obstructive sleep apnea that occurs with almost 83% of cardiovascular disease, 58% of heart failure and 53% of atrial fibrillation, to name a few.

The system 300 can also be used for users that snore, but who do not yet have sleep apnea. The inclusion of the vibration and airflow sensor enables the measurement of the intensity of snoring and can open the airway before the sub-sonic snore has become audible. The inclusion of stimulators of soft palate and uvula can reduce or eliminate snoring in users that do not have sleep apnea yet. Also, the system 300 can be used along with a CPAP machine and enable the CPAP machine to be used at a lower air pressure than a typical setting for user's that cannot tolerate CPAP machine at their typical air pressure.

In one example, the devices disclosed herein are worn by a user at nighttime and includes sensors to monitor nocturnal silent angina or myocardial ischemia (measured by continuous EKG monitoring) that could cause sudden death or acute myocardial infarction during sleep or wake (especially silent ischemia). With the medical dispenser present, an incident could be treated with release of sublingual nitroglycerine from medicament reservoir while data such as continuous blood pressure recording, EKG, echocardiography and carotid doppler blood flow is continuously recorded and transmitted to the controller station 200 or cloud server 300. The cloud server 300 can then send the data to a monitoring on-call physician, a handheld device or computer to alert the patient, as well to the nearest ER/ED (emergency room) for early ambulance dispatch.

In an athletic environment, the sensors selected for use in the maxillary and mandibular devices disclosed herein can be the pulse-oximetry, CDT/CNT based infra-red oxygenation receptors, heart rate and EKG, PTT with non-invasive blood pressure recording, carotid blood flow, CMUT/IVUS doppler ultrasound, blood glucose level (in tongue or soft palate) for diabetic or hyperglycemic individual, airway resistance and total tidal volume (airflow measurement per breath), EEG recording, respiratory rate measurement, core body temperature, temperature and humidity derived from respiratory (inspiratory and expiratory) airflow, computational mini-Incentive Spirometry based on above inspiratory-expiratory airflow or time ratio (early detection of exercise-induced asthma in an athlete, a soldier or a fitness or weight loss buff), and combinations thereof. Data from these sensors will allow determination of performance restrictions and methods to physiologically improve performance such as legal nutritional supplementation or medications, such as aerosolized asthma medication or aerosolized insulin for a diabetic athlete, soldier or a fitness or weight loss buff, for underlying medical conditions or increasing the size of airway to help improve oxygenation and reduce heart rate, reduce elevations of body temperature or loss of humidity during exercise or athletic performance.

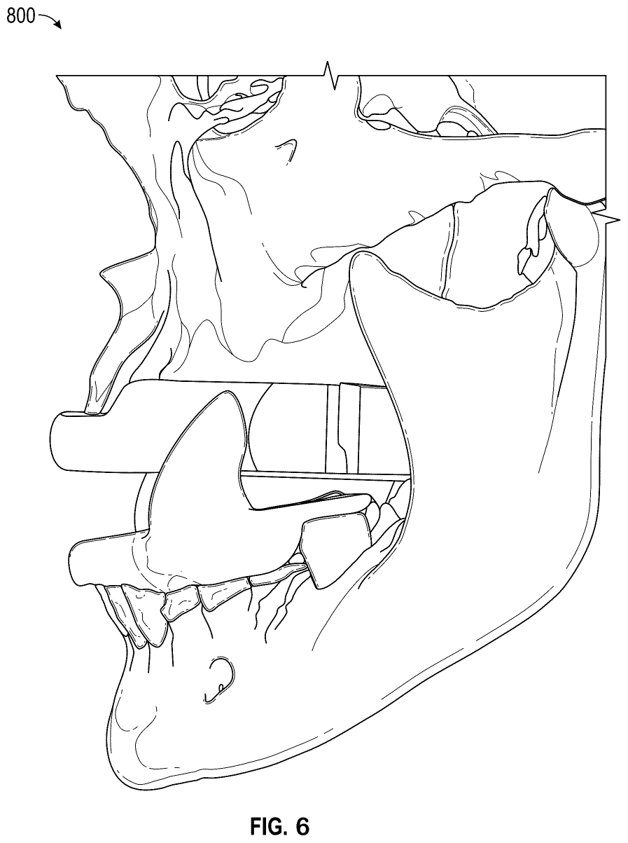

The first ten minutes after a concussion are extremely important in preventing brain injuries. A concussed athlete loses consciousness, muscles relax, and thereafter, the tongue and jaw fall back and obstruct the airway. As shown in FIG. 6, the anterior to posterior advancement and cranial to caudal advancement of the mandibular repositioning device 800 (also referred to as an anterior vertical mandibular lingual repositioning device (AVMLRD)) disclosed herein holds the jaw and hence the tongue forward and down relative to the maxillary piece, which improves opens the airway (increases the size of the airway) for improved airflow, breathing and oxygenation of the brain and heart (along with other organs). For a concussed athlete wearing an AVMLRD, another person needs to place two hands behind the jaw and using two thumbs gently move the chin down, thereby the AVMLRD automatically draws the jaw and tongue forward creating a bigger airway and clearing an obstruction thereto. This can save an athlete's life and reduce brain injury resulting from loss of oxygen. This positive effect will be further incrementally improved upon with electrical stimulation of the tongue (when a lingual stimulator is installed in the AVMLRD) to aid in moving the tongue forward. The AVMLRD can reduce the recovery period, reduce long-term damaging consequences of concussions, and prevent traumatic brain injury (TBI) and chronic traumatic encephalopathy (CTE). Sensors useful for application to concussions that can be included in the AVMLRD include EEG sensors, carotid doppler blood flow ultrasound sensor, airway SMCA and airflow sensors. Sensors could also be present in the helmet or other gear of the athlete, including those just mentioned as well as accelerometers and/or GPS location. Sensors can transmit information regarding airway closure, brain waves, and/or oxygen levels to the system, which can trigger the F-AI and/or P-AI algorithms to implement active opening of the user's airway when the stimulator and/or motorized or robotic drivers are present in the AVMLRD. Moreover, the system enables the supervising manager of the user to monitor the user's data from the digital platform and can notify emergency medical systems or teams to be on standby or to intervene.

In one aspect, methods of lowering heart rate during physical activity are disclosed. The method starts by identifying a person having a smaller than normal SMCA (discussed in the background section) in need of being increased while awake. Most people are not aware of the size of their smallest concentric airway cross-sectional area, and many people have a SMCA that is ⅓ of the normal size average of 149 mm². These people often think that their athletic performance or ability to lose weight is limited by their talent or effort, but it may actually have a direct correlation to the size of their SMCA. Breathing is simply less efficient for these people and the increased effort to breath causes early fatigue of the diaphragm and the abdominal-thoracic muscle of respiration and drives the heart rate up to the cardiovascular workout rate rather than remaining at the fat burning rate. Increase in exercise causes an increase in body temperature, heart rate, and respiratory rate. Hear rate increases by 10 beats per minute for every 1° C. increase in body temperature. Women are inherently more prone to early fatigue because of a natural tendency to have smaller SMCA, thereby demonstrating greater hypoxemia than the same size man with similar height and build. This device and method, therefore, has the potential to help women improve their fitness, weight, and thus impact their overall health in a positive manner.

Next, the identified person is provided with a mandibular repositioning device fitted for their respective mandible and maxilla that has a maxillary piece comprising a tooth covering having a driver flange protruding laterally outward on a right side proximate a backmost teeth mold and/or on a left side proximate a backmost teeth mold, each driver flange having an anterior side with a convex curvature and a mandibular piece comprising a tooth covering having a protrusive flange extending cranially therefrom positioned to have a posterior side engaged with the anterior side of each driver flange, the posterior side of each protrusive flange has a concave-to-convex curvature from its base toward its most cranial point and a convex portion of the concave-to convex curvature engages the convex curvature of the driver flange in a rest position. In such a mandibular repositioning device downward movement of the mandibular piece moves the convex portion of the posterior side of the protrusive flange along the convex curvature of the driver flange, thereby moving a user's mandible forward. During physical activity, simply by opening the mouth, the mandibular repositioning device advances the mandible caudally and anteriorly, whereby it increases the size of the person's smallest concentric airway cross-sectional area making breathing easier (decrease the resistance to airflow during breathing), increasing airflow, decrease (or preventing an undesired increase) said person's heart rate for a given level of exercise, decrease in CO₂ level, increase oxygen saturation, decrease in relative respiratory rate for a given level of exercise, reduction in generation of excessive body heat at a given exercise level, and decrease loss of water from a reduction in sweating and through respiration (respiratory rate), decrease loss of electrolytes, decrease in muscle cramps, increased endurance, increased speed, increased stamina, increased strength during exercise, increase in physical performance.

The mandibular repositioning devices disclosed herein by increasing the smallest concentric airway cross-sectional area of a user's airway, which is behind the tongue, works for both mouth breathing and nose breathing. The improvements in breathing (respiratory rate) from decreases airflow resistance achieved by the anterior-posterior repositioning and cranial to caudal repositioning yield the additional benefit of maintaining body water content (decreasing the amount of dehydration relative to the given exercise) and lowering the rate of rise in body temperature, both of which improve endurance.

In one embodiment, the physical activity is athletic or military training in which data from sensors included in the mandibular repositioning device are monitored by a coach or superior to determine or monitor how anterior-posterior and cranial-caudal repositioning adjustments effect the user and various parameters such as heart rate, body temperature, respiratory rate, oxygen saturation level, etc. In particular, the height of the open mouth in the cranial-caudal direction can be “dialed in” to maximize endurance or another aspect of the user's performance. This may be determined by monitoring the user's physical activity while making incremental changes in the anterior-posterior and cranial-caudal repositioning adjustments. In another embodiment some sensors are in the oral appliance while others may be on the athlete/trainee's skin, inside a wristwatch-type wearable biometric sensing device or other bodily biometric sensors that are all feeding data into the controller station or hand-held smart device that is being monitored by the coach or supervisor.

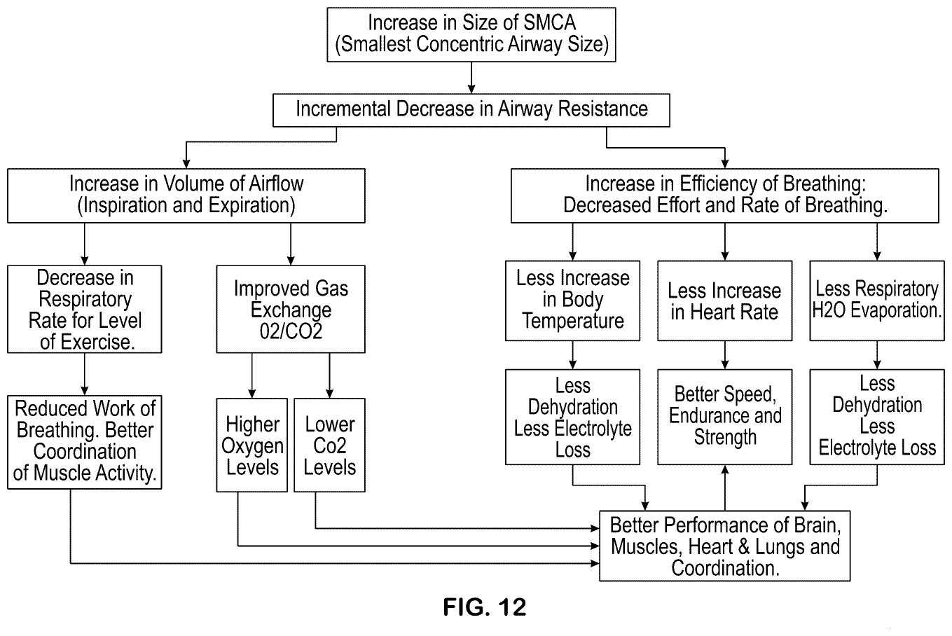

The above is equally applicable to weight loss activities. Determining the user's anterior-posterior and cranial-caudal repositioning adjustments for fat burning activity is of great importance. Most individuals that try to exercise for the purpose of losing weight give up because of unsatisfactory results over a short period of time. Due to the body's deconditioned state, heart rate rises rapidly into the cardio range with light exercise. This prevents the individual from losing weight although they do get cardio exercise. Moreover, carrying excess weight causes increased oxygen consumption. The individual is unable to increase oxygen delivery due to a limited capacity to breathe. This is a limitation of the narrowest cross-sectional area in their airway (SMCA) posterior to the tongue. Under normal circumstances, an individual simply has no choice but to breath harder to bring in more oxygen. This increases work of breathing, increased body fluid loss, sweating, increased body heat, increased heart rate and quicker fatigue. Thus, resulting in termination of the workout and eventually majority of individuals give up the training. AVMLRD can increase the narrowest cross-sectional area (SMCA) that is the limiting factor. With reference to FIG. 12, incremental reduction in airflow resistance and increase in airflow and oxygenation with lesser work of breathing will delay dehydration, sweating, body heat rise, fatigue and keep the heart rate at a lower level (within the fat burning range) while offering higher amount of calorie consumption for a longer period of time, thus increasing the possibility of successful weight loss. It is also expected that users will experience an improvement in personal self-image, emotional markers and mental health, and possibly an increase in endorphins and decrease in adrenaline production during exercise and better control of diabetes and blood sugar problems.

System 300 can be used for scheduled timed administration of medication through the mechanisms and devices discussed above, especially for those medications best administered while the user is asleep. When medicaments are being administered by the devices disclosed herein, the controller station 200 or system 300 would identify a physiological problem of the user from data received from the sensors and/or from data received from an external EKG monitoring system or external blood-glucose monitoring system of the user followed by generation of an executable instruction sent to the device's on-board microprocessor through wireless data system (blue tooth or other protocols) with back-up confirmation system for dangerous medications. The back-up may be the user themselves (smart phone or display screen of controller Station 200) or an on-call nurse or ER physician or authorized health care provider or tele-medicine through a smart handheld device or through videography/audio from a camera or video recorder in the mandibular or maxillary housing. Data related to administration of the medication would require a response the following day prompting replacement of discharged pellets or other forms of the medicament, a visit to the health care provider's office, or a tele-medicine visit.

The mandibular repositioning devices disclosed herein with their ability to increase the dimensions of the smallest concentric airway cross-sectional area will be able to be used in the field of pediatrics, adult cardiology, adult pulmonology, adult endocrinology, and metabolism, adult gastro-enterology, adult neurology and sleep, adult rheumatology, adult hematology and oncology, adult ophthalmology, and adult nephrology. In pediatrics, the devices can improve or reduce problems caused by asthma, vasomotor rhinitis, pediatric obstructive sleep apnea, cystic fibrosis, bronchiectasis, tracheomalacia, gastro-esophageal reflux, hiatal hernia, recurrent URI, recurrent tonsillitis, adeno-tonsillar hypertrophy, bruxism, hypoplastic palate retrognathia, ADD/ADHA, childhood obesity, failure to thrive, learning difficulty, depression, epilepsy, headaches, nightmares, night-terrors, sleep-walking nighttime bedwetting, pediatric hypertension, Duchene's Muscular Dystrophy, Becker's Muscular Dystrophy, Spino-muscular atrophy, facio-scapulo-humeral dystrophy, and Marfan's Syndrome. In adults, the devices can improve or reduce problems caused by hypertension, coronary artery disease, congestive heart failure, left ventricular hypertrophy, diastolic dysfunction, right ventricular hypertension, mitral regurgitation, tricuspid regurgitation, aortic regurgitation, aortic stenosis, supra-ventricular tachycardia, ventricular tachycardia, atrial fibrillation, and atrial flutter, premature atherosclerosis, atrial enlargement, ventricular enlargement, asthma, COPD, emphysema, bronchiectasis, pulmonary fibrosis, pulmonary embolism, acute respiratory failure, ventilator weaning program management in ICU or rehabilitation, cardio-pulmonary rehabilitation, aspiration pneumonia, obesity, hypothyroidism, diabetes mellites, hyperclolesterolemia, osteoporosis, gastro-esophageal reflux, esophageal stricture, hiatal hernia, gall bladder disease, gall stones, non-alcoholic steatosis of the liver, non-alcoholic cirrhosis, irritable bowel syndrome, embolic and thrombotic stroke, cerebral hemorrhage due to rupture of aneurysm, cluster headaches, migraines, periodic limb movement disorder of sleep, restless leg syndrome, nightmares, night terrors, REM sleep behavior disorder, dementia, Alzheimer's disease, neurodegenerative disease like Parkinson's, Ley Body disease, chronic or acute inflammatory demyelinating polyneuropathy, seizures, PTSD, myasthenia gravis, fibromyalgia, RA, SLE, Barrett's esophagus, esophageal cancer, secondary polycythemia, myelodysplastic syndrome, glaucoma, retinal vein occlusion, tortuosity of retinal veins, retinal artery disease, retinal detachment, macular degeneration, acute retinal stroke, chronic renal failure, benign and malignant nephrosclerosis, renal artery stenosis, fibromuscular dysplasia of renal artery, and nocturia.

Moreover, using the controller station 200 and cloud server of the system 300, it will be possible to receive data regarding the user's input of food and time consumed to act proactively during sleep based on a correlation of digestion time and acid reflux onset. This capability may be extended to input of any and all medications, physiological data such as BP, EKG and blood sugar, and to administering of any and all medications during the day (prompted to the user through handheld device) or night (automatically performed if pressure pellet for medication is available to the system to discharge sub-lingually or intra-orally in liquid form or inhaled as micro-aerosol powder form.

Referring now to FIG. 13, Algorithm I and the results for one individual as example are shown. Algorithm I predict the total anterior vertical mandibular lingual repositioning adjustments for effective use of the mandibular repositioning devices herein with a user during physical activities, such as weight loss, athletics, military, and aerospace activities.

The baseline heart rate is the heart rate at rest for a selected user. The target hear rate is the heart rate that is selected by the user or a professional assisting the user with the physical activity such as a coach, fitness expert, doctor, physical therapist, etc. For example, a heart rate that is less than 70% of the peak heart rate may be desired for fat burning activities and can be used in a weight loss program. The peak heart rate is a variable that is dependent upon the age of the selected user and is not gender specific.

Airflow resistance is determined by setting a fixed length of airway to 10 cm and the air viscosity to 1.81 (typical for normal elevations). An increase in airflow resistance causes an increase in heart rate, respiratory rate, work of breathing, and core body temperature, and affects calorie or energy consumption, water loss through sweat, and evaporation through breathing.

Referring now to FIG. 14, Algorithm II and results for one individual as an example are shown. The use of Algorithm II for physical activity monitoring, such as during a sporting activity, is meant to be used to improve the individual's performance, such as work efficiency, lower heart rate, etc. while mitigating risk of deterioration from other indices, such as dehydration, core body temperature, etc. The actual exercise hear rate is measured during a preselected activity. In one embodiment, the preselected activity is running on a treadmill at 4 miles per hour while at a 5% incline. The change in heart rate is important for determining the progress of the individual at a fitness program or physical activity. Monitoring the incremental increase/decrease in the heart rate with adjustment of TAVMLR or intensity of exercise provides a new method of monitoring an individual. The physical activity can be adjusted for the individual at any given time including adjustment of the TAVMLR for the mandibular repositioning devices disclosed herein to create an optimal size of SMCA for the level of exercise desired at that time.

The respiratory rate is a determinant of the work of breathing, which is dependent on oxygen consumption, demand, and cardio-pulmonary coupling. The faster an individual is breathing, the greater the work being performed and the earlier the individual will be fatigued. It also determines the generation of body heat and amount of water evaporation (dehydration). The minute ventilation (MV) has a proportion relationship to the respiratory rate. However, with increasing respiratory rate there is also increase in dead space ventilation and the proportion of alveolar ventilation for each breath begins to decrease. Increasing inefficiency of breathing (the greater the respiratory rate) generates greater lactic acidosis which clouds consciousness and decision making, produces easy muscle cramps with deterioration in coordination, and creates higher heart rates. It is very important to keep the minute ventilation (respiratory rate) in a manageable (in a range so as to maintain greatest efficiency of breathing or lowest airflow resistance) range to optimize the individual's work, i.e., minimize effort and maximize performance.

The air pressure or airway pressure is the pressure generated by forceful inhalation and exhalation. It is reflective of the work of breathing but also is a determinant of airway resistance. It may cause the airway to be sucked inward (Bernoulli's effect) during inspiration which decreases the SMCA further and deteriorates performance. An increase in pressure may precipitate exercise induced asthma. The work of breathing, typically, needs to be maintained at <10% of the basal metabolic rate (BMR) for an endurance athlete. Greater values are reflective of greater calorie consumption and may be desirable for weight loss programs. These algorithms help optimization of work of breathing to suit the intended or desired result.

Rising core body temperature can be a source of dehydration and rapid fatigue. It may also generate conditions like heat stroke and all its associated complications like rhabdomyolysis, in endurance athletes. The closer this value is to baseline, the safer for the individual. This value is dependent on heart rate and airway resistance, with the airway resistance being dependent on the size of SMCA. Each heart rate increase of 10 beats per minute increases the core body temperature by 1° C.

Dehydration is expressed in the Algorithms as milliliters per hour. Many individuals fail to complete an activity because of poor management of dehydration. Dehydration is also a factor in injuries suffered during physical activity. Muscle weakness and cramping and decreasing blood pressure due to decreasing intravascular volume are important complications of dehydration. Water loss is inevitable. Understanding the anticipated volume of water loss and replacing it judiciously is now possible because the algorithms estimate the volume of water loss.

An individual that experiences a decrease in the systolic blood pressure (SBP) with exercise is referred to as a “Dipper.” Such a decrease in the SBP is a predictor of cardiovascular risk of myocardial infarction and cardiac arrhythmia. A severe increase in SBP is associated with exercise related cardiac complications like stroke and cardiac arrhythmia. Algorithm II enables the SBP to be monitored, thereby enabling the identification of either fluctuation in the SBP for timely intervention and/or prevention of such complications during physical activity. The double product (DP) output of Algorithm II is the SBP multiplied by the pulse rate. This numerical value is used in stress tests as an index of myocardial oxygen consumption. A safe range for DP is 14,000 to 18,000. DP can be monitored by Algorithm II during physical activity to prevent severe increases and decreases in cardiac oxygen consumption. For Dippers, the Algorithms provide a TAVMLR value that when implemented in a mandibular repositioning device that is worn during physical activity can result in a healthy increase in SBP while reducing the heart rate using better provision of oxygen through an increased SMCA, thus keeping the DP within a safe range. Target Peak SBP=1.2*Resting SBP and the same goes for target DBP. In a Dipper, Target Peak SBP=0.8*Resting SBP. The multiplier 1.2 represents a 20% increase in SBP that occurs in normal individuals while the multiplier 0.8 represents the 20% drop in SBP from resting value.

Methods that include the algorithms are numerous. The method can determine the SMCA without requiring a CT Cone Scan of an individual's airway, can determine the TAVMLR to manufacture a customized mandibular repositioning device of the kinds disclosed herein to improve an individual's performance during physical activity, can determine and/or prevent a risk of a cardiac event during exercise, can build fitness programs to fulfill or satisfy a large range of variables and individual needs, can set incremental targets for any of the variables in either algorithm to increase physical performance, such as speed, endurance, ability to jump higher or longer, swim faster, can set incremental targets for any of the variables to reduce the TAVMLR in order to purposefully increase airway resistance to build greater endurance or decrease/increase airflow resistance to mimic conditions of extremely high or low G forces in space or in a military or civilian aircraft during distress, can predict target outcomes and set nutritional supplementation and hydration levels to maximize the success of such targets, can protect athletes and individuals from medical conditions like asthma, unsafe high or low levels of SBP or DBP, can be used in medical practice to adjust the TAVMLR to ease breathing or optimize cardiac performance in conditions like, but not limited to, cystic fibrosis, bronchiectasis, COPD and CHF, Angina, or to treat obesity or diabetes.

The methods include determining baseline parameters for a selected individual, the baseline parameters comprising age, baseline heart rate, respiratory rate, core body temperature, and minute ventilation while at rest, determining the air pressure for an exercise location of the individual, selecting a target heart rate for a preselected physical activity, and calculating the total anterior and vertical mandibular lingual repositioning (TAVMLR) for a mandibular repositioning device using one or more of Algorithms I and II. In one embodiment, the TAVMLR is calculated according to the equation:

$$TAVMLR = 0.1839 * ( 3.142 * {( ( 8 * {V}_{a} * {L}_{airway} ) / {( ( target heart rate - baseline heart rate ) / ( the air pressure * 100 ) )}^{1 / 4} )}^{2} )$$

wherein Vₐ is the viscosity of air=1.81 and L_{airway} is the length of the airway=10 cm. The TAVMLR value determines the amount of anterior repositioning and vertical repositioning to include in the mandibular repositioning device of the selected user for an at rest position of the mandible.

For Algorithm II, the method includes measuring the individual's actual exercise heart rate at a preselected activity and intensity. In one embodiment, the preselected activity and intensity is a run on a treadmill at 4 miles per hour at a 5% incline. In another embodiment, it is a brisk walk on the treadmill instead of a run. In another embodiment, it may be a brisk walk at 5 miles per hour at an 8% incline. The duration of the preselected activity may very as part of the intensity, such as being for a half hour or 45 minutes, whatever the overseer of the physical activity deems appropriate for the individual to establish an exercise heart rate that fits the ultimate goal of the method.

The methods can include reviewing the output parameters of Algorithm II for any outputs that are outside of a desired range for the individual or for making intensity adjustments to the physical activity for the individual to prevent injury, medical emergencies, etc., and gradual changes in intensity (increases in particular) can be projected and implemented. For example, the double product is outside of the range of 14,000 to 18,000, the blood pressure is too high or too low for the individual, the heart rate is too high for fat burning exercise, etc.

In one embodiment, the individual has their blood pressure monitored while exercising for continual live updating of the Algorithms, especially Algorithm II. The live updating can be accomplished with a mobile device, computer, or other screen capable of displaying the data generated by the algorithms. If the individual is one whose systolic blood pressure (SBP) drops with exercise, e.g., SBP changes from 160 mm Hg to 128 mm Hg, the overseer of the exercise/physical activity can monitor this in real time and can terminate the exercise or reduce the intensity as needed. Software can connect the biometric parameters with the exercise equipment and even control the exercise parameters on the associated equipment (like treadmill or a stationary bicycle) such as slowing or speeding up the treadmill in response to the algorithm. The overseer of the physical activity may even create exercise scenarios to push the physiologic parameter outside of the normal range to increase the endurance or tolerance during physical activity of the individual for example so as to create conditions of severe stress in order to condition the individual to be properly conditioned for the physical activities demands. The individual as mentioned before can be an athlete, military personnel, astronaut, pilot, etc.

Moreover, the overseer can adjust the variables INPUT or OUTPUT in the Algorithms as needed to tailor suitable exercise conditions for individuals with pre-existing conditions. Some example conditions include, but are not limited to, asthma, COPD, cardiac arrhythmia, and excessive sweating syndrome. For example, if the individual has Asthma, the Desired Air Pressure can be selected to stay under 1.32 because expiratory airway pressure rises with intensity of exercise can precipitate an attack of exercise induced asthma. In this example, we still want the Target % Peak HR to stay close to 55-60%. Using the Goal Seek tool in the Excel program, we set the Target Airway Pressure to 1.32 and the algorithm then creates the scenario for safe athletic training at 1.32.

The systems disclose herein have numerous advantages, including the digital platform which enables an authorized supervising manager with capability to monitor and record patient data in real time, learn the patient, and alter the patient's treatment in real-time.

A unique advantage of this system over any other existing systems is that the jaw and tongue can move synchronously, independently, or sequentially during sleep or during wake-related activities, in real-time and in anticipation of impending airway closure or changes in physiology, and in a provision of a measured response to those changes such as relief from, restriction of airflow as determined by the controller station 200 even before the airway has completely closed; thus, restoring unrestricted airflow even before the patient has completely stopped breathing (as in sleep apnea). This system can see airway obstruction before it happens and will keep the airway constantly open in any body position or depth of sleep. This is a distinct advantage over CPAP/BIPAP or any other mechanical or electrical system that is commercially available in the market. In addition, there are distinct advantages just by the breadth of functionality that has been described above.

The App and digital platform include learning algorithms in the memory of the microprocessor that learns a user's sleep patterns and other physiological events and functions during sleep and wake, pathological events and activities during wake and sleep from the data collected over time and creates a “best response” for the simultaneous, independent, or sequential responses exemplified by tensing of the soft palate or Uvula, release of medication or stimulation of the stimulator and activation of the first and second drivers to open the airway or to train muscles of speech, and to synchronize these best responses such as exemplified by certain jaw movements that are associated with particular phases of respiration.

The device and system disclosed herein have numerous advantages, including artificial intelligence utilizing data collected by the MLRD during use to actively in real-time adjust the MLRD in response to the phases of respiration, degree of obstruction of the airway, snore sounds and vibrations and amount of hypoxemia present relative to each breath irrespective of the stage of sleep of the user, various levels of exertion, physical activity, and other applications. The system is capable of measuring a large number of cardiac, neurological and endocrine sensory inputs as described above exemplified by continuous non-invasive glucose, oxygen, blood pressure, pH monitoring, heart rhythm and temperature etc. The system is capable of photography for creating dental impressions, dentures or to diagnose gum disease etc. The system is capable of executing a large spectrum of functions such as mandible protrusion, administering sub-lingual insomnia medication like Intermezzio or cardiac medication like nitroglycerine or training muscle groups for swallowing or speech. The system is capable of communicating with user, provider, EHR (Electronic Health Record) and pharmacy etc. This system is capable of determining restriction to airflow, increase in velocity of air and turbulence, decreasing levels of oxygen and increasing levels of heart rate, pH monitoring and any other physiological parameter that could be installed in the future with constant inputs of physiological parameters (unlike with CPAP machine or oral appliances that are available in the industry), such as those mentioned above. This 24 hour a day seven days a week capability of collection and processing of data allows the system to actually make adjustments exemplified by the movement of the mandible and tongue prior to closure of the airway and hence will work as a preventative form of treatment for sleep apnea.

It should be noted that the embodiments are not limited in their application or use to the details of construction and arrangement of parts and steps illustrated in the drawings and description. Features of the illustrative embodiments, constructions, and variants may be implemented or incorporated in other embodiments, constructions, variants, and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention. Having described the invention in detail and by reference to preferred embodiments thereof, it will be apparent that modifications and variations are possible without departing from the scope of the invention which is defined in the appended claims.

1. A system comprising:
a computing application implementable on a user's personal computing device, the computing application comprising:
downloadable software comprising a plurality of modules and instructions, wherein the plurality of modules includes a first module configured for entry of a user's biometrics, a second module configured for entry of a user's health and/or fitness goals, a third module configured for calculating a plurality of variables relative to the user's health and/or fitness goals, and a fourth module configured for operable electronic communication with a managing platform;
wherein the plurality of variables are selected from the group consisting of carbohydrates daily intake, protein daily intake, fat daily intake, calories per day, calories per week, basal metabolic rate, ml/hr hydration loss during exercise, active metabolic rate, a target calorie burn rate, and combination thereof; and
the managing platform is configured for operative electronic communication with the computing application over a communication network, the managing platform comprises a user interface configured for implementing a plurality of modules and instructions;
wherein the plurality of modules comprises a fifth module configured for managing the data from the user's computing application, a sixth module configured for managing access of a supervising manager to date from the user's computing application, a seventh module configured for managing one or more algorithms executable by the third module of the computing application.

2. The system of claim 1, wherein the fourth module of the downloadable software is configured to be activatable and de-activatable by the user.

3. The system of claim 1, wherein the user wears a mandibular repositioning device comprising:
a maxillary piece configured to cover teeth of a user comprising a right side backmost teeth mold and/or a left side backmost teeth mold and a driver flange protruding laterally outward on either or both of the right side and left side backmost teeth mold, each driver flange having an anterior side with a convex curvature; and
a mandibular piece comprising:
a tooth covering having a protrusive flange extending cranially therefrom positioned to have a posterior side engaged with the anterior side of each driver flange, the posterior side of each protrusive flange has a concave-to-convex curvature from a base of the protrusive flange toward a most cranial point of the protrusive flange and a convex portion of the concave-to convex curvature engages the convex curvature of the driver flange in a rest position.

4. The system of claim 3, wherein the mandibular piece has a plateau of a preselected height between the base of the protrusive flange and the tooth covering, wherein the preselected height is set to prevent disconnect between each protrusive flange and a respective driver flange relative to a fully open mouth measurement between incisors of the user.

5. The system of claim 3, wherein the one or more algorithms and user data cooperate operatively within the third module to iteratively evaluate the variables configuring the protrusive flange and the driver flange of the user, wherein the output of the third module is a recommended change to one or more of the variables of the user's protrusive flange or driver flange.

6. The system of claim 5, wherein the variables are selected from the group consisting of curvature, height, width, lean, thickness, yaw, rotation, position on the mandible piece or maxillary piece, position of sensors.

7. The system of claim 5, wherein the recommended change is set to incrementally increase or decrease a health or fitness parameter of the user.

8. The system of claim 7, wherein the health or fitness parameter is selected from the group consisting of air flow volume, air flow rate, oxygen levels, glucose levels, blood pressure, heart rate, respiratory rate, core body temperature elevation during a physical activity, release of aerosolized medications, inhalation of aerosolized medications, running speed, and blood sugar values.

9. The system in claim 1, wherein the supervising manager of the user has access to the one or more algorithms to adjust variables to determine a desired health or fitness parameter or one or more of the variables of the protrusive flange and the driver flange.

10. The system of claim 1, wherein the supervising manager is selected from the group consisting of a dentist, a medical professional, a health professional, an athletic trainer, an athletic manager, a weight loss trainer, a physical fitness trainer, an athletic coach, a dietician, and combinations thereof.
